(19) United States
(12) Patent Application Publication
(10) Pub. No.: US 2026/0315499 A1
(43) Pub. Date: Oct. 15, 2026

(54) 2'- {HETEROCYCLYL (ARYL/ALKYL) METHYL} - CANNABIDIOL COMPOUNDS AND PROCESS FOR PREPARATION THEREOF

(71) Applicant: COUNCIL OF SCIENTIFIC & INDUSTRIAL RESEARCH, New Delhi, Delhi (IN)

(72) Inventors: Pankaj Singh CHAM, Jammu (IN); Loveleena ANAND, Jammu (IN); Yassir ARFATH, Jammu (IN); Nadia RASHID, Jammu (IN); Mir Shahid MAQBOOL, Jammu (IN); Ria GUPTA, Jammu (IN); Zaheen AKHTER, Jammu (IN); Varun Pratap SINGH, Jammu (IN); Zabeer AHMED, Jammu (IN); Sheikh Rayees RAFIQ, Jammu (IN); Fayaz Ahmad MALIK, Jammu (IN); Parvinder Pal SINGH, Jammu (IN)

(21) Appl. No.: 19/482,973

(22) PCT Filed: May 10, 2024
(86) PCT No.: PCT/IN2024/050516

§ 371 Date: Nov. 10, 2025

(30) Foreign Application Priority Data
May 11, 2023 (IN) 202311033447

(51) Int. Cl. A61K 31/00; A61P 25/00; A61P 29/00; C07D 211/14; C07D 211/18; C07D 211/46; C07D 239/42; C07D 241/20; C07D 295/096; C07D 295/135; C07D 295/185

(52) U.S. Cl. CPC A61K 31/658; A61P 25/00; A61P 29/00; C07D 211/14; C07D 211/18; C07D 211/46; C07D 239/42; C07D 241/20; C07D 295/096; C07D 295/135; C07D 295/185

(57) ABSTRACT

In a ring annulated cannabidiol compound, either one or both the alpha position to OH in aromatic ring is attached to the hetero-ring through spacer. Furthermore, the ring annulated cannabidiol can be used as a neuroprotective and anti-inflammatory agent.

### FIELD OF INVENTION

The present invention relates to cannabidiol compounds as anti-depression and anti-inflammatory agents. The present invention particularly relates to analogues of cannabidiol, where either one or both the alpha position to OH in aromatic ring is attached to a hetero-ring through spacer. The present invention more particularly relates to the method of preparation of such analogues of cannabidiol and their use as anti-depression and anti-inflammatory agents.

### BACKGROUND OF THE INVENTION

Depression being a complex phenomenon is associated with feelings of dejection and dependency. It is a frequent serious disorder in the general population and is one of the biggest health challenges the world faces today (Indian J Psychol Med., 2010, 32(1), 1-2; Psychiatr Danub., 2018, 30(3), 273-284).

Major depressive disorder (MDD) ranks among the ten leading causes of disability worldwide targeting 3.8% of population among which women are the major sufferers. According to WHO, MDD affects approximately 350 million people worldwide with 15.9% prevalence in the Indian subcontinent. (Psychol Med., 2018, 48(9), 1560-1571).

In spite of scientific understanding and many therapeutic discoveries in last few decades, there is no significant improvement in the therapeutic options available for depression-related disorders (J Affect Disord., 2009, 116(1-2), 4-11).

Most treatment advancements over the past 50 years have been directed at enhancing monoamine transmission by blocking serotonin (5-HT) and nor-epinephrine (NE) transporters. However, a significant number of patients do not respond to these treatments. Conventional therapeutic options continue to have limitations, including the pharmacological side effects, long treatment timeframes, and most significantly partial efficacy (or reduced functionality). There is an urgent unmet need of new therapy in this domain either as new chemical entities or modified version of available therapies with better safety index.

Cannabinoids (endogenic, phytogenic, and synthetic) with anti-depressant and anti-inflammatory properties have been well-documented and studied, and have been reviewed recently (Front Pharmacol., 2017, 8, 1-18; Bioorg. Med. Chem., 2015, 23, 1377-1385).

Although their anti-depressive mechanism of action is not entirely known, multiple studies have highlighted the importance of a number of receptors and pathways that are connected to neurological disorders, proliferative inflammation, and severe depression. The most important receptors for the anti-depressant and anti-inflammatory potential are G protein-coupled receptors such as CB1, CB2 and GRP55, transient receptor potential channels such as TRPV and TRPA, FAAH (fatty acid amide hydrolase), NAPE-PLD (N-acyl phosphatidylethanolamine phospholipase D), MAGL (monoacylglycerol lipase), DAGL (diacylglycerol lipase) etc. (Int. J. Mol. Sci., 2021, 22(11), 6152; Mol Brain., 2017, 10(1):38). Cannabidiol (CBD), a USFDA approved antiepileptic drug has also been extensively researched for its anti-depressant and anti-inflammatory properties.

CBD has demonstrated anti-depressant activity against major depressive disorders such as bipolar disorder, anxiety, post-traumatic stress disorder and schizophrenia. It has also shown anti-inflammatory action against inflammatory disorders such as inflammatory bowel disease, rheumatoid arthritis, ear inflammation, neuro-inflammation and lung inflammation. These studies demonstrated significant therapeutic potential of cannabidiol as an anti-depressant and anti-inflammatory agent, but low oral bio-availability and poor drug-like properties are some of the major associated concerns (Front Pharmacol., 2018, 9, 1365; Br. J. Clin. Pharmacol., 2018, 84 (11), 2477-2482).

Therefore, further work is still needed to develop next-generation candidates with superior profiles, target engagement, and drug-likeness. In this direction, the current invention disclosed the synthesis of cannabidiol analogues as anti-depressant and anti-inflammatory agents.

Several cannabidiol analogues have been designed, synthesized and patented for diverse pharmacological applications, however, the approaches as well as application are different from the present invention. The literature examples are also highlighted here:

WO2021/062559, describes the carboxamide-based cannabidiol derivatives but it differs from the presently synthesized cannabidiol derivatives containing alpha position to OH in aromatic ring are attached to the hetero ring.

WO2021/062557, describes the carboxamide-based tetrahydrocannabinol derivatives but it differs with respect to cannabidiol derivatives synthesised herein, which contain alpha position to OH in aromatic ring are attached to the hetero ring.

WO2021/000054, describes the dioxinonyl-based cannabinoid derivatives for the treatment and prevention of diseases such as acute pain, ADHD/ADD, alcohol use disorder, allergic asthma, ALS, Alzheimer's, anorexia etc.

### OBJECTIVE OF THE INVENTION

The main object of the present invention is to provide the analogues of cannabidiol.

Another object of the present invention is to provide a process for the synthesis of such analogues of cannabidiol by using Mannich reaction with secondary amine and primary amine.

Yet another object of the present invention is to use such analogues of CBD as anti-depression and anti-inflammatory agents.

### SUMMARY OF THE INVENTION

Accordingly, the present invention provides cannabidiol compounds of Formula A and stereoisomers thereof,

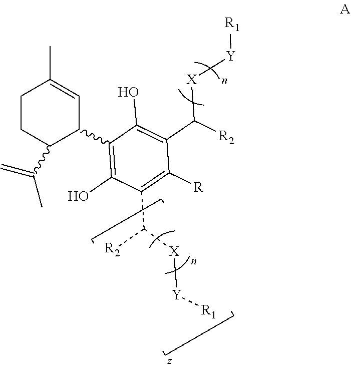

wherein X is independently selected from C and N;
Y is selected from heterocycle, and heteroaryl;
n and z are integers independently selected from 0 and 1;
R is selected from H, OH, protected hydroxyl, alkyl, alkenyl, alkynyl, acyl, aryl, heteroaryl, cycloalkyl and heterocycle, wherein the alkyl, alkenyl, alkynyl and acyl group is optionally substituted with one or more groups, independently selected from the group consisting of halogen, —OH, alkyl, —O-alkyl, NR′R″, S-alkyl, alkynyl, aryl, heteroaryl, cycloalkyl and heterocycle, and the aryl or heteroaryl group is optionally substituted with one or more substituents selected from the group consisting of halogen, —OH, alkyl, —O-alkyl, —COOH, —C(O), —C alkyl, —C(O)OC and NR′R″; wherein R′ and R″ are independently selected from the group consisting of H, alkyl, alkenyl, alkynyl and acyl;
R₁ is independently selected from the group consisting of H, OH, alkyl, alkenyl, alkynyl, acyl, aryl, heteroaryl, cycloalkyl and heterocycle, wherein the alkyl, alkenyl, alkynyl and acyl group is optionally substituted with one or more groups, independently selected from the group consisting of halogen, —OH, alkyl, —O-alkyl, NR′R″, S-alkyl, —SO-alkyl, —SO₂-alkyl, aryl, S-aryl, —SO-aryl, —SO₂-aryl, —SO₂—N-aryl, —N—SO₂-arylalkenyl and alkynyl; aryl, heteroaryl, cycloalkyl and heterocycle is optionally substituted with one or more substituents selected from the group consisting of halogen, OH, alkyl, —O-alkyl, —COOH, —C(O), —C alkyl, —C(O)OC, NR′R″; wherein R′ and R″ are independently selected from the group consisting of H, alkyl, alkenyl, alkynyl and acyl;
R₂ is selected from H, alkyl, alkyl aryl and aryl;

In a preferred embodiment of the present invention Y in the compound of Formula A is selected from the group consisting of morpholinyl, piperidinyl, piperazinyl, pyrazinyl, pyrrolidinyl, tetrahydrofuranyl, pyridinyl, pyrimidinyl, phenyl and benzyl.

In a preferred embodiment of the present invention R₁ in the compound of Formula A is selected from the group consisting of:

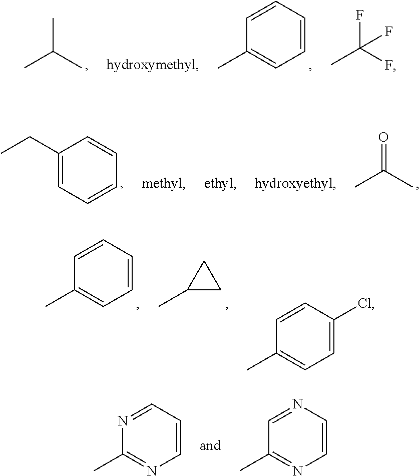

In a preferred embodiment of the present invention the cannabidiol is selected from the group consisting of:

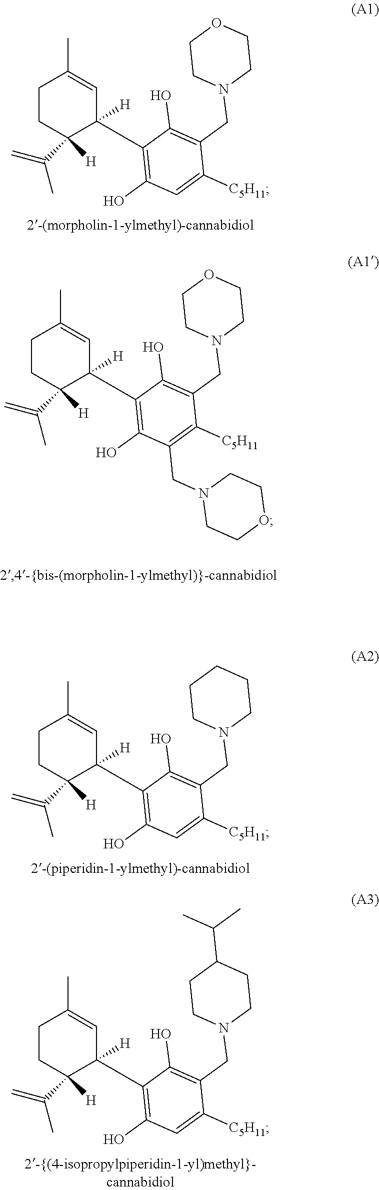

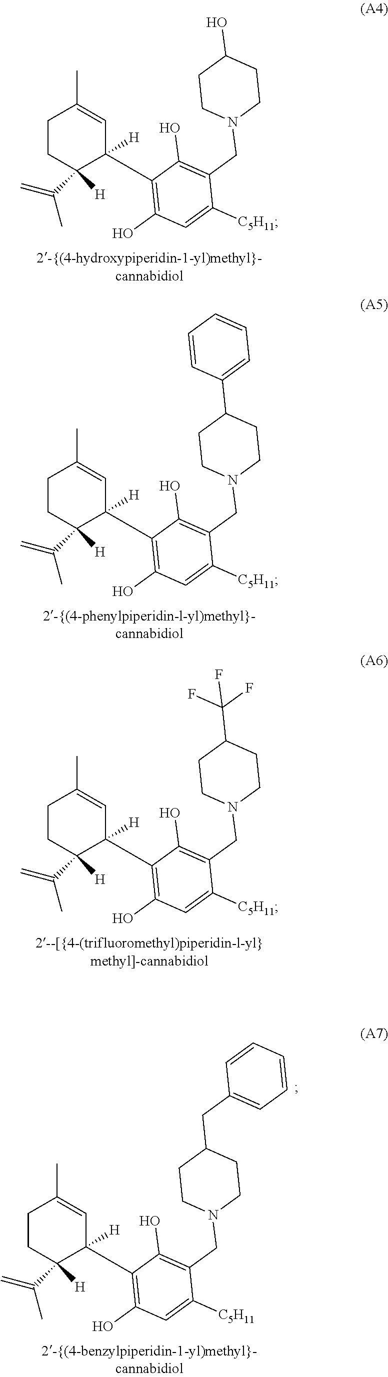

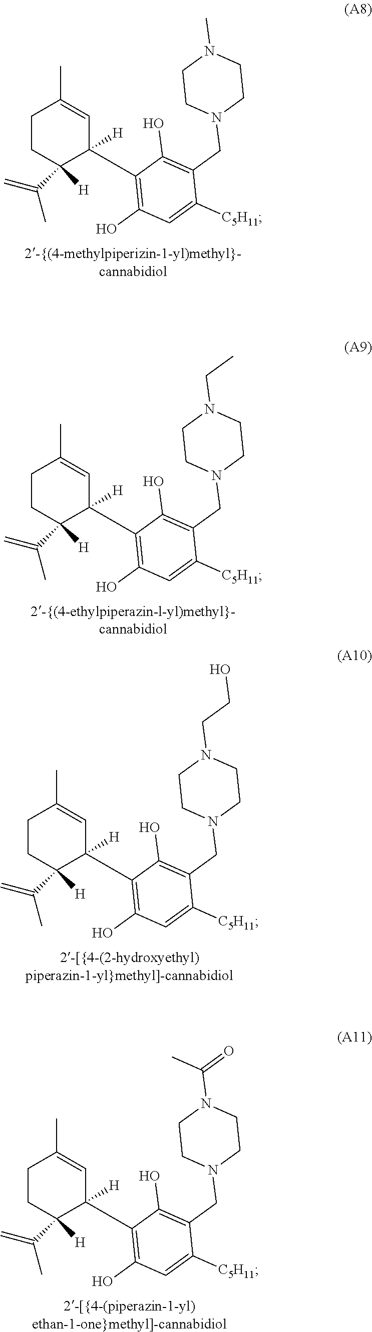

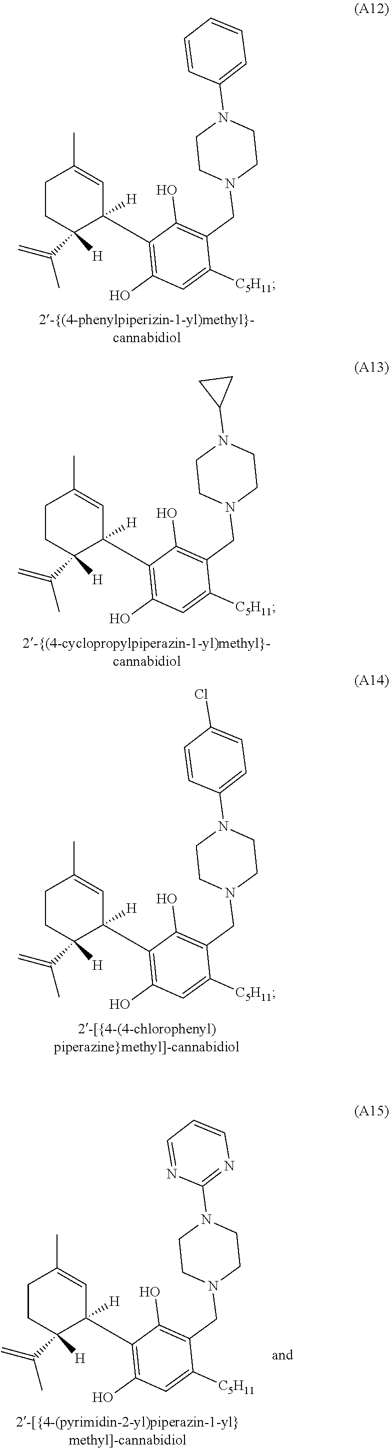

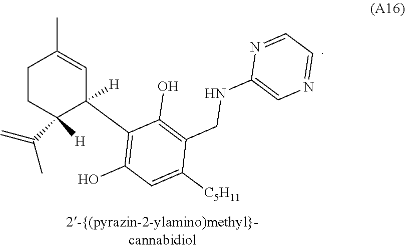

In an embodiment of the present invention the compound has two stereo-centers which may be R, S or a mixture of both.

The present invention also provides a process for the synthesis of ring annulated cannabidiol compound of Formula A and stereoisomers thereof,

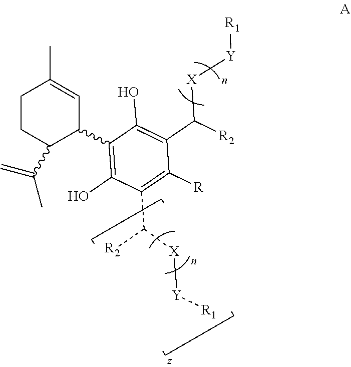

wherein X is independently selected from C and N;
Y is selected from heterocycle and heteroaryl;
n and z are integers independently selected from 0 and 1;
R is selected from H, OH, protected hydroxyl, alkyl, alkenyl, alkynyl, acyl, aryl, heteroaryl, cycloalkyl and heterocycle, wherein the alkyl, alkenyl, alkynyl and acyl group is optionally substituted with one or more groups, independently selected from the group consisting of halogen, —OH, alkyl, —O-alkyl, NR′R″, S-alkyl, alkynyl, aryl, heteroaryl, cycloalkyl and heterocycle, and the aryl or heteroaryl group is optionally substituted with one or more substituents selected from the group consisting of halogen, —OH, alkyl, —O-alkyl, —COOH, —C(O), —C alkyl, —C(O)OC and NR′R″; wherein R′ and R″ are independently selected from the group consisting of H, alkyl, alkenyl, alkynyl and acyl;
R₁ is independently selected from the group consisting of H, OH, alkyl, alkenyl, alkynyl, acyl, aryl, heteroaryl, cycloalkyl and heterocycle, wherein the alkyl, alkenyl, alkynyl and acyl group is optionally substituted with one or more groups, independently selected from the group consisting of halogen, —OH, alkyl, —O-alkyl, NR′R″, S-alkyl, —SO-alkyl, —SO₂-alkyl, aryl, S-aryl, —SO-aryl, —SO₂-aryl, —SO₂—N-aryl, —N—SO₂-arylalkenyl and alkynyl; aryl, heteroaryl, cycloalkyl and heterocycle is optionally substituted with one or more substituents selected from the group consisting of halogen, OH, alkyl, —O-alkyl, —COOH, —C(O), —C alkyl, —C(O)OC, NR′R″; wherein R′ and R″ are independently selected from the group consisting of H, alkyl, alkenyl, alkynyl and acyl;
R₂ is selected from H, alkyl, alkyl aryl and aryl.
comprising the steps of:
   i. coupling of fragment B, wherein R is as defined above and fragment C, wherein R₁ is as defined above in the presence of an aldehyde D, wherein R₂ is as defined above at a temperature in the range of 25-30° C., for a period in the range of 13-16 hours optionally in presence of a solvent and

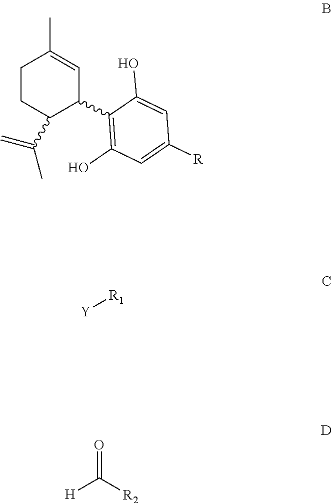

ii. purifying the obtained compound of formula A.

In a preferred embodiment of the present invention, the solvent used in the process is selected from the group consisting of dichloromethane, chloroform, isopropanol, acetone, acetonitrile methanol or a combination thereof.

In a preferred embodiment of the present invention, the fragment C is selected from the group consisting of:

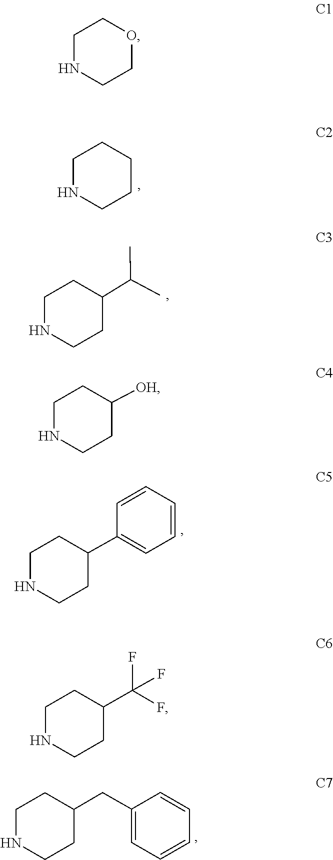

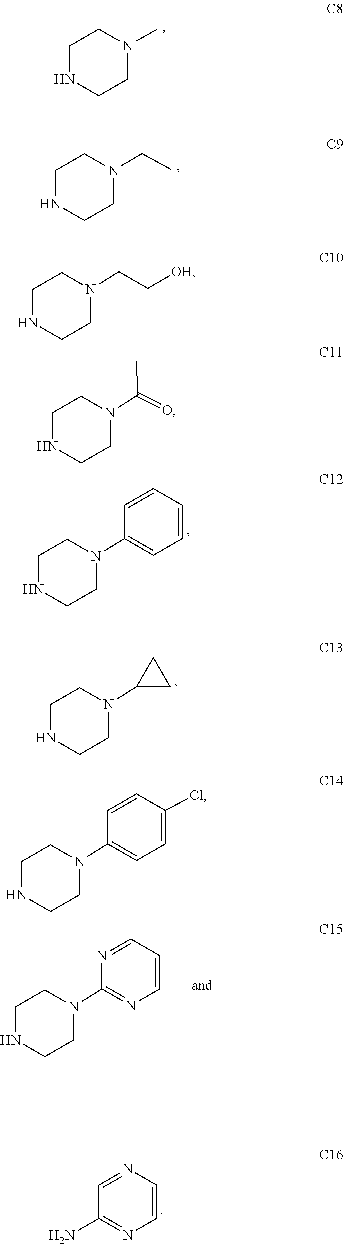

In a preferred embodiment of the present invention, the aldehyde D is selected from the group consisting of formaldehyde, propionaldehyde, acetaldehydes, propionaldehyde, butyraldehyde, isovaleraldehyde, benzaldehyde and cinnamaldehyde.

In another aspect, this invention provides the compounds exhibit neuroprotective properties in neuronal cell lines of human and mice origin.

In another aspect, this invention provides the compounds exhibit neuroprotection activity in neuronal cell lines in concentration and time dependent manner.

In another aspect, this invention provides the compounds enhanced the expression of survival pathway proteins.

In another aspect, this invention provides the compounds exhibit neuroprotection against the corticosterone induced depression in animal model.

In another aspect, this invention provides the compounds exhibit anti-inflammatory activity by inhibiting the NO production.

In another aspect, this invention provides the compounds exhibit cell viability potential in macrophages.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### List of Abbreviations

1) ADHD/ADD—Attention Deficit Hyperactivity Disorder
2) ALS—Amylotropic Lateral Sclerosis
3) ATCC—American Type Culture Collection
4) BDNF—Brain Derived Neurotrophic Factor
5) CB1—Cannabinoid Receptor Type 1
6) CBD—Cannabidiol
7) CDCl₃—Deuterated Chloroform
8) CD₃OD—Deuterated Methanol
9) CHCl₃—Chloroform
10) CH₃OH—Methanol
11) DAGL—Diacylglycerol Lipase
12) DEPT—Distortionless Enhancement by Polarization Transfer
13) DHF—Dihydroxy Flavone
14) DMEM—Dulbecco's Modified Eagle Medium
15) ESI-TOF—Electrospray Ionization Time-of-Flight
16) FAAH—Fatty Acid Amine Hydroxylase
17) FBS—Fetal Bovine Serum
18) GRP55—Protein-Coupled Receptor 55
19) HT—5-hydroxytryptamine
20) NMR—Nuclear Magnetic Resonance
21) H₂O—Water
22) HRMS—High-Resolution Mass Spectrometry
23) LC-MS—Liquid Chromatography-Mass Spectrometry
24) LPS—Lipopolysaccharide
25) MDD—Major Depressive Disorder
26) MeOH—Methanol
27) MHz—Megahertz
28) MS—Mass Spectrometry
29) MTT—(3-(4,5-dimethylthiazol-2-yl)-2,5-diphenyltetrazolium Bromide
30) NAPE-PLD—N-acyl Phosphatidylethanolamine Phospholipase D
31) N2A—Mouse Neuroblastoma Cells
32) NE—Nor Epeinephrine
33) nm—Nanometre
34) NMR—Nuclear Magnetic Resonance
35) NO—Nitric Oxide
36) OD—Optical Density
37) P-ErK—Phospho Extracellular Signal-Regulated Kinase
38) P-AkT—Phospho AkT
39) PPM—Parts Per Million
40) RAW264.7—Mouse Monocyte/Macrophage Cell Line
41) RBF—Round Bottom Flask
42) SHSY5Y—Human Neuroblastoma Cell Line
43) TLC—Thin Layer Chromatography
44) TOF—Turnover Frequency
45) TrkB—Tyrosine Receptor Kinase B
46) TRPA—Transit Receptor Potential Ankyrin
47) TRPV—Transit Receptor Potential Vanniloid
48) TST—Tail Suspension Test
49) UHD—Ultra High Definition
50) USFDA—United States Food and Drug Administration

### DETAILED DESCRIPTION OF THE INVENTION

For a better understanding of the invention, a detailed description of the invention along with detailed description of the preferred embodiments of the present invention is explained below with reference to the accompanying tables and drawings.

For better understanding the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “consisting of” means the embodiment necessarily includes the listed components only and no other unlisted components are present. Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. The term “between” should be understood as being inclusive of the limits.

The term “alkyl” refers to a saturated hydrocarbon chain having the specified number of carbon atoms. In an aspect of the present disclosure, alkyl refers to an alkyl group having 1-10 carbon atoms. Alkyl groups may be straight or branched chained groups which may be optionally substituted. Representative branched alkyl groups have one, two, or three branches. Preferred alkyl groups include, without limitation, methyl, ethyl, n-propyl, and isopropyl. One or more hydrogens of the alkyl groups may be optionally replaced with deuterium such as CD₃, CHD₂, CDH₂, CH₂CD₃, CD₂CH₃ and the like.

The term “alkenyl” refers to an unsaturated hydrocarbon chain having a specific number of carbon atoms and at least one double bond between carbon atoms. In an aspect of the present disclosure, alkenyl refers to an alkenyl group having 2-10 carbon atoms. Alkenyl groups may be straight or branched chained groups. Representative branched alkenyl groups have one or two, branches. Examples of alkenyl groups include, but not limited to, ethenyl, n-propenyl, and isopropenyl.

The term “alkynyl” refers to an unsaturated hydrocarbon chain having a specific number of carbon atoms and at least one triple bond between carbon atoms. In an aspect of the present disclosure, alkynyl refers to an alkynyl group having 2-10 carbon atoms. Alkynyl groups may be straight or branched chained groups which may be optionally substituted. Representative branched alkyl groups have one, two, or three branches. Examples of alkynyl groups include, but not limited to, ethynyl, n-propynyl, or butynyl.

The term “acyl” refers to a functional group comprising a carbonyl group attached with an organic carbon group of the formula R^{a}—C═O, wherein R^{a} is selected from alkyl, alkenyl, or alkynyl which are optionally substituted.

The term “cycloalkyl” used herein refers to a saturated non-aromatic carbocyclic ring with 3 to 10 carbon atoms, may be optionally substituted by one or more substituents. Examples of cycloalkyl includes but not limited to cyclopropyl, cyclobutyl, cyclopentyl, cyclohexyl.

The term “heterocycle”, “heterocyclic” or “heterocyclyl” refer to saturated or unsaturated rings containing specific number of carbon atoms in a range of 3 to 7 with one or more heteroatoms and the heterocyclic group may be monocyclic, bicyclic, or polycyclic. The heterocyclic group may be fused, bridged, or spiral structures and may be optionally substituted. In certain embodiments, ‘heterocycle’ groups are saturated. In other embodiments, ‘heterocycle’ groups are unsaturated. In few embodiments, ‘heterocycle’ groups are partially unsaturated. ‘Heterocycle’ groups containing more than one heteroatom may contain different heteroatoms selected from N, S, and O. ‘Heterocycle’ groups may be substituted with one or more substituents as defined herein. ‘Heterocycle’ includes oxetanyl, morpholinyl, pyrrolidinyl, piperazinyl, piperidinyl, tetrahydropyranyl, azepinyl, oxazepinyl, azabicyclo[3.1.0]hexanyl.

The term “heteroaryl” refers to an aromatic heterocyclic ring radical as defined above. The heteroaryl ring radical may be attached to the main structure at any heteroatom or carbon atom resulting in the creation of a stable structure. The heteroaryl refers to an aromatic ring with one or more hetero atoms selected from N, O or S with carbon ranging between 2 to 10.

In the context of the present invention, the term “optionally” or “optional” when used in reference to any element, to intermediates, reagents or conditions, including any method step, e.g., the isolation of intermediates; is intended to mean that the subject element is isolated, or alternatively is not isolated from the reaction mixture and directly used for the subsequent chemical reaction. Similarly, this definition is applied in case for reagents or reaction conditions as well.

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively and any and all combinations of any or more of such steps or features.

The present invention discloses the cannabidiol compounds of Formula A and stereoisomers thereof,

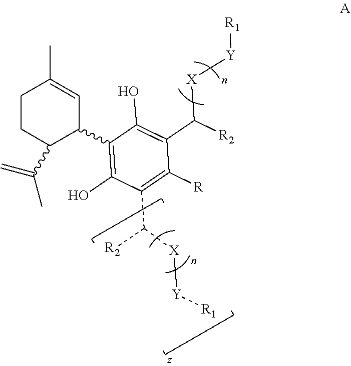

wherein X is independently selected from C and N;
Y is selected from heterocycle, or heteroaryl;
n and z are integers independently selected from 0 and 1;
R is selected from H, OH, protected hydroxyl, alkyl, alkenyl, alkynyl, acyl, aryl, heteroaryl, cycloalkyl or heterocycle, wherein alkyl, alkenyl, alkynyl or acyl group is optionally substituted with one or more groups, independently selected from the group consisting of halogen, —OH, alkyl, —O-alkyl, NR′R″, S-alkyl, alkynyl, aryl, heteroaryl, cycloalkyl and heterocycle, and aryl or heteroaryl group is optionally substituted with one or more substituents selected from the group consisting of halogen, —OH, alkyl, —O-alkyl, —COOH, —C(O), —C alkyl, —C(O)OC and NR′R″; wherein R′ and R″ are independently selected from the group consisting of H, alkyl, alkenyl, alkynyl and acyl;
R₁ is independently selected from the group consisting of H, OH, alkyl, alkenyl, alkynyl, acyl, aryl, heteroaryl, cycloalkyl and heterocycle, wherein the alkyl, alkenyl, alkynyl and acyl group is optionally substituted with one or more groups, independently selected from the group consisting of halogen, —OH, alkyl, —O-alkyl, NR′R″, S-alkyl, —SO-alkyl, —SO₂-alkyl, aryl, S-aryl, —SO-aryl, —SO₂-aryl, —SO₂—N-aryl, —N—SO₂-arylalkenyl and alkynyl; aryl, heteroaryl, cycloalkyl and heterocycle is optionally substituted with one or more substituents selected from the group consisting of halogen, OH, alkyl, —O-alkyl, —COOH, —C(O), —C alkyl, —C(O)OC, NR′R″; wherein R′ and R″ are independently selected from the group consisting of H, alkyl, alkenyl, alkynyl and acyl;
R₂ is selected from H, alkyl, alkyl aryl and aryl.

In another embodiment of the present disclosure, X is independently selected from C or N; Y is selected from heterocycle, or heteroaryl; n selected from 0 or 1; z is 0; R is selected from H, OH, alkyl, alkenyl, alkynyl, aryl, heteroaryl, cycloalkyl or heterocycle, wherein the alkyl, alkenyl, or alkynyl group is optionally substituted with one or more groups, independently selected from the group consisting of halogen, —OH, alkyl, —O-alkyl, NR′R″, S-alkyl, alkynyl, aryl, heteroaryl, cycloalkyl and heterocycle; and aryl or heteroaryl group is optionally substituted with one or more substituents selected from the group consisting of halogen, —OH, alkyl, —O-alkyl, —COOH, —C(O), —C alkyl, —C(O)OC and NR′R″; wherein R′ and R″ are independently selected from the group consisting of H, alkyl, alkenyl, alkynyl and acyl; R₁ is independently selected from the group consisting of H, OH, alkyl, alkenyl, alkynyl, acyl, aryl, heteroaryl, cycloalkyl or heterocycle, wherein alkyl, alkenyl, alkynyl or acyl group is optionally substituted with one or more groups, independently selected from the group consisting of halogen, —OH, alkyl, —O-alkyl, NR′R″, S-alkyl, —SO-alkyl, —SO₂-alkyl, aryl, S-aryl, —SO-aryl, —SO₂-aryl, —SO₂—N-aryl, —N—SO₂-arylalkenyl and alkynyl; and aryl, heteroaryl, cycloalkyl and heterocycle is optionally substituted with one or more substituents selected from the group consisting of halogen, OH, alkyl, —O-alkyl, —COOH, —C(O), —C alkyl, —C(O)OC, NR′R″; wherein R′ and R″ are independently selected from the group consisting of H, alkyl, alkenyl, alkynyl and acyl; and R₂ is selected from H, alkyl, alkyl aryl or aryl.

In yet another embodiment of the present disclosure, X is independently selected from C or N; Y is selected from heterocycle, or heteroaryl; n selected from 0 or 1; z is 0; R is selected from H, OH, or alkyl, wherein alkyl group is optionally substituted with one or more groups, independently selected from the group consisting of halogen, —OH, alkyl, —O-alkyl, NR′R″, S-alkyl, alkynyl, aryl, heteroaryl, cycloalkyl and heterocycle; R₁ is independently selected from the group consisting of H, OH, alkyl, acyl, aryl, heteroaryl, cycloalkyl or heterocycle, wherein alkyl, or acyl group is optionally substituted with one or more groups, independently selected from the group consisting of halogen, —OH, alkyl, —O-alkyl, NR′R″, S-alkyl, —SO-alkyl, —SO₂-alkyl, aryl, S-aryl, —SO-aryl, —SO₂-aryl, —SO₂—N-aryl, —N—SO₂-arylalkenyl and alkynyl; and aryl, heteroaryl, cycloalkyl or heterocycle is optionally substituted with one or more substituents selected from the group consisting of halogen, OH, alkyl, —O-alkyl, —COOH, —C(O), —C alkyl, —C(O)OC, NR′R″; wherein R′ and R″ are independently selected from the group consisting of H, alkyl, alkenyl, alkynyl and acyl; and R₂ is selected from H, or alkyl.

In yet another embodiment of the present disclosure, X is N; Y is selected from C₃₋₇ heterocycle or C₃₋₇ heteroaryl; n selected from 0 or 1; z is 0; R is C₁₋₆ alkyl, wherein C₁₋₆ alkyl group is optionally substituted with one or more groups, independently selected from the group consisting of halogen, —OH, C₁₋₆ alkyl, —O-C₁₋₆ alkyl, NR′R″, S—C₁₋₆ alkyl, C₂₋₆ alkynyl, C₂₋₆ aryl, C₃₋₇ heteroaryl, C₃₋₇ cycloalkyl and C₃₋₇ heterocycle; R₁ is independently selected from the group consisting of H, OH, C₁₋₆ alkyl, C₁₋₆ acyl, C₆₋₁₀ aryl, C₃₋₇ heteroaryl, C₃₋₇ cycloalkyl or C₃₋₇ heterocycle, wherein C₁₋₆ alkyl, or C₁₋₆ acyl group is optionally substituted with one or more groups, independently selected from the group consisting of halogen, —OH, C₁₋₆ alkyl, —O—C₁₋₆ alkyl, NR′R″, S—C₁₋₆ alkyl, —SO—C₁₋₆ alkyl, —SO₂—C₁₋₆ alkyl, C₆₋₁₀ aryl, S—C₆₋₁₀ aryl, —SO—C₆₋₁₀ aryl, —SO₂—C₆₋₁₀ aryl, —SO₂—N-aryl, —N—SO₂—C₇₋₁₀ arylalkenyl and C₂₋₆ alkynyl; and C₆₋₁₀ aryl, C₃₋₇ heteroaryl, C₃₋₇ cycloalkyl or C₃₋₇heterocycle is optionally substituted with one or more substituents selected from the group consisting of halogen, OH, C₁₋₆ alkyl, —O— C₁₋₆ alkyl, —COOH, —C(O), —C₁₋₆ alkyl, —C(O)OC, NR′R″; wherein R′ and R″ are independently selected from the group consisting of H, C₁₋₆ alkyl, C₂₋₆ alkenyl, C₂₋₆ alkynyl and acyl; and R₂ is selected from H, or C₁₋₆ alkyl.

There are two stereo-centre in the molecules, wherein ~~~ represents a single bond it may be above the plane or below the plane. The stereo-centre may be R, S as well as mixture of both.

In an embodiment of the present disclosure, there is provided a cannabidiol compound of Formula A as disclosed herein, wherein Y is selected from the group consisting of morpholinyl, piperidinyl, piperazinyl, pyrazinyl, pyrrolidinyl, tetrahydrofuranyl, pyridinyl, pyrimidinyl, phenyl and benzyl. In another embodiment of the present disclosure, Y is selected from the group consisting of morpholinyl, piperidinyl, pyridinyl, piperazinyl, and pyrazinyl.

In an embodiment of the present disclosure, there is provided a process for the synthesis of cannabidiol compounds of Formula A,

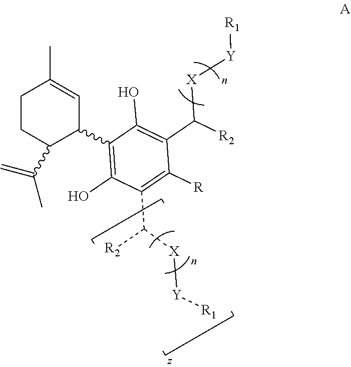

wherein R, R₁ and R₂ are as defined above, comprises of:
coupling of fragment B and fragment C in the presence of aldehyde fragment D;

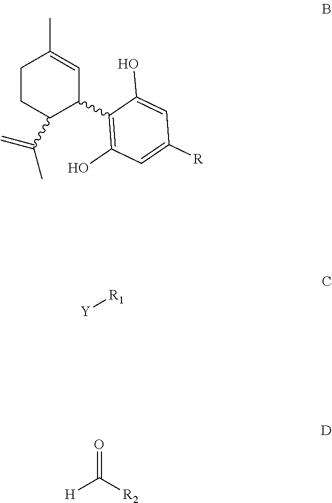

wherein R, R₁ and R₂ in B, C and D respectively are as defined above; and purifying the obtained compound. The coupling is carried out in presence of a solvent at a temperature in the range of 25-30° C., for a period in the range of 13-16 hours.

There are two stereo-centres in the molecules, wherein ~~~ represents a single bond it may be above the plane or below the plane. The stereo-centre may be R, S as well as mixture of both.

A preferred embodiment of the present invention relates to the synthesis of cannabidiol compounds of Formula A by the coupling of fragment B and fragment C in the presence of aldehyde fragment D in a solvent as provided in Scheme 1.

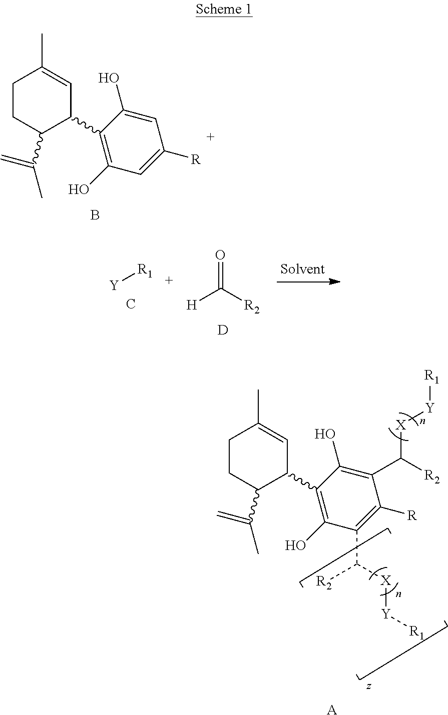

R, R₁ and R₂ in B, C and D respectively are as defined above. The aldehyde D is selected from the group consisting of formaldehyde, propionaldehyde, acetaldehydes, propionaldehyde, butyraldehyde, isovaleraldehyde, benzaldehyde, cinnamaldehyde either alone or in combination thereof. The solvent is a single solvent or a mixture of one or more solvents and the solvent is selected from the group consisting of dichloromethane, chloroform, isopropanol, acetone, acetonitrile methanol either alone or in combination thereof.

A preferred embodiment of the present invention relates to the synthesis of a compound of Formula A1 namely 2′-(morpholin-1-ylmethyl)-cannabidiol and Formula A1′ namely 2′,4′-{bis-(morpholin-1-ylmethyl)}-cannabidiol by the coupling of fragment B1 namely cannabidiol and fragment C1 namely morpholine in the presence of fragment D1 in a solvent.

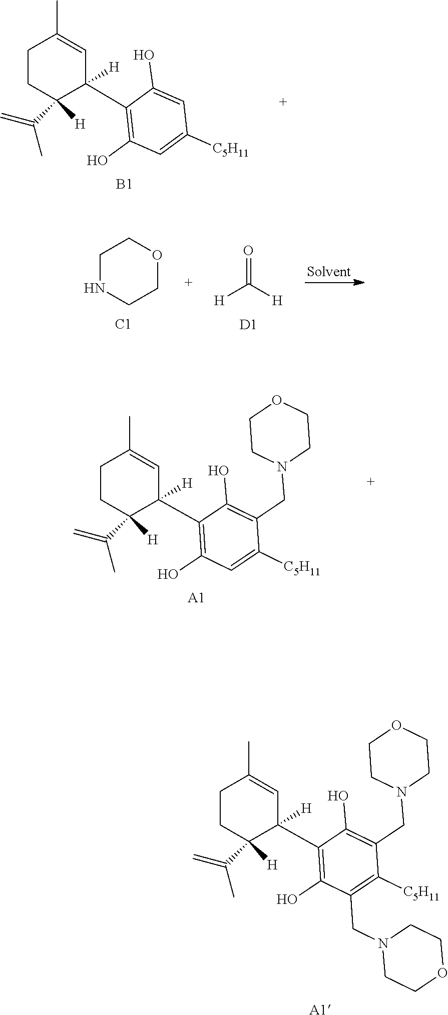

Another preferred embodiment of the present invention relates to the synthesis of a compound of Formula A2 namely 2′-(piperidin-1-ylmethyl)-cannabidiol by the coupling of fragment B1 namely cannabidiol and fragment C2 namely piperidine in the presence of fragment D1 in a solvent.

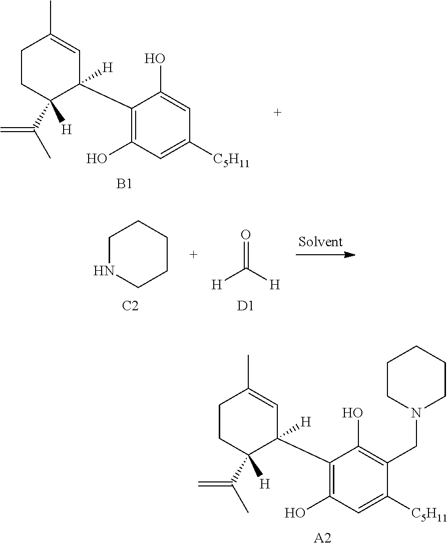

The present invention relates to the synthesis of a compound of formula A3 namely 2′-{(4-isopropylpiperidin-1-yl)methyl}-cannabidiol by the coupling of fragment B1 namely cannabidiol and fragment C3 namely 4-isopropylpiperidine in the presence of fragment D1 in a solvent.

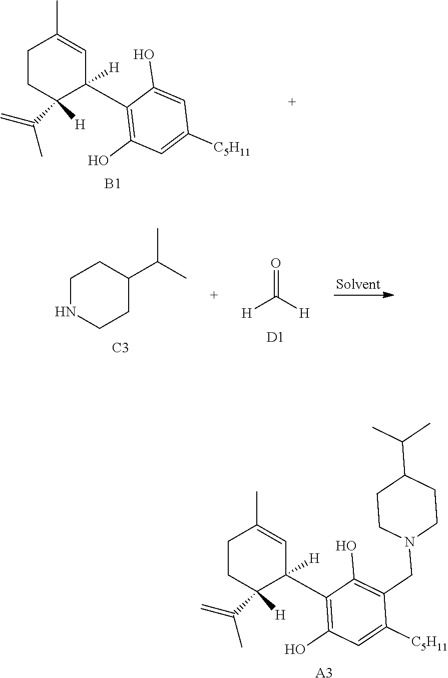

The present invention relates to the synthesis of a compound of formula A4 namely 2′-{(4-hydroxypiperidin-1-yl)methyl}-cannabidiol by the coupling of fragment B1 namely cannabidiol and fragment C4 namely piperidin-4-ol in the presence of fragment D1 in a solvent.

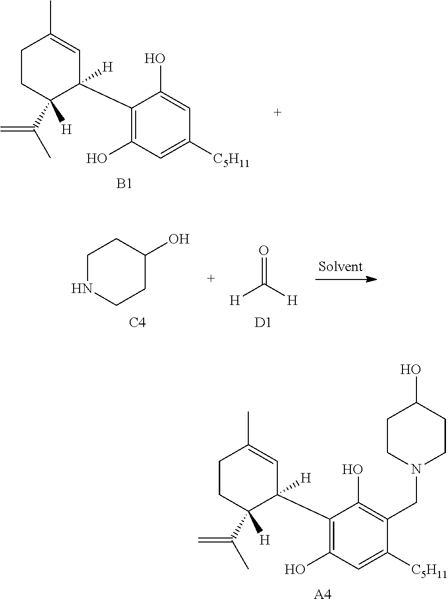

The present invention relates to the synthesis of a compound of formula A5 namely 2′-{(4-phenylpiperidin-1-yl)methyl}-cannabidiol by the coupling of fragment B1 namely cannabidiol and fragment C5 namely 4-phenylpiperidine in the presence of fragment D1 in a solvent.

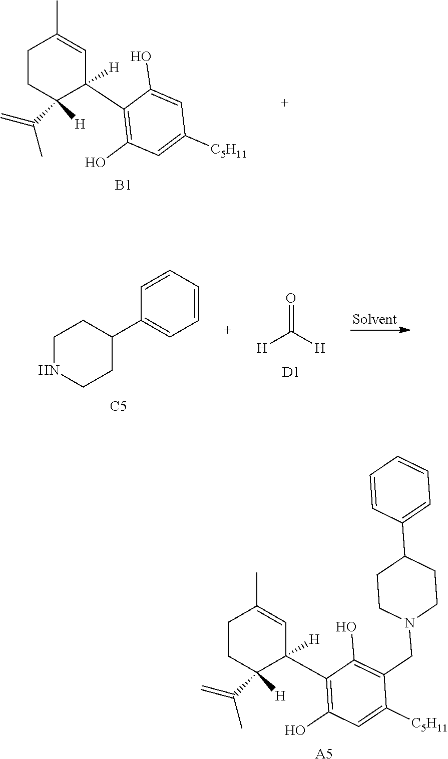

The present invention relates to the synthesis of a compound of formula A6 namely 2′-[{4-(trifluoromethyl)piperidin-1-yl}methyl]-cannabidiol by the coupling of fragment B1 namely cannabidiol and fragment C6 namely 4-(trifluoromethyl)piperidine in the presence of fragment D1 in a solvent.

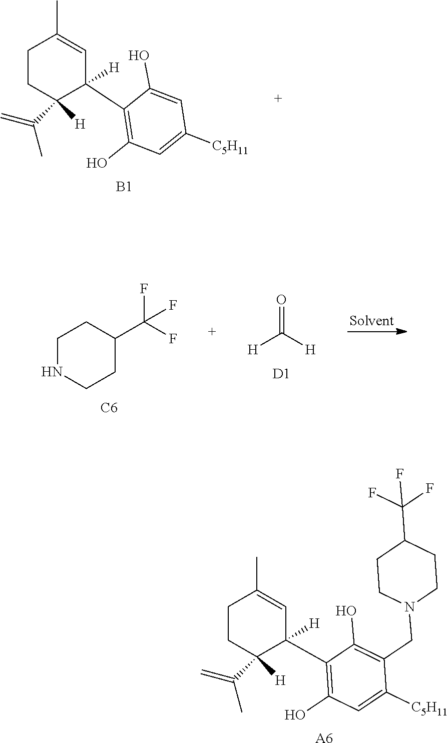

The present invention relates to the synthesis of a compound of formula A7 namely 2′-{(4-benzylpiperidin-1-yl)methyl}-cannabidiol by the coupling of fragment B1 namely cannabidiol and fragment C7 namely 4-benzylpiperidine in the presence of fragment D1 in a solvent.

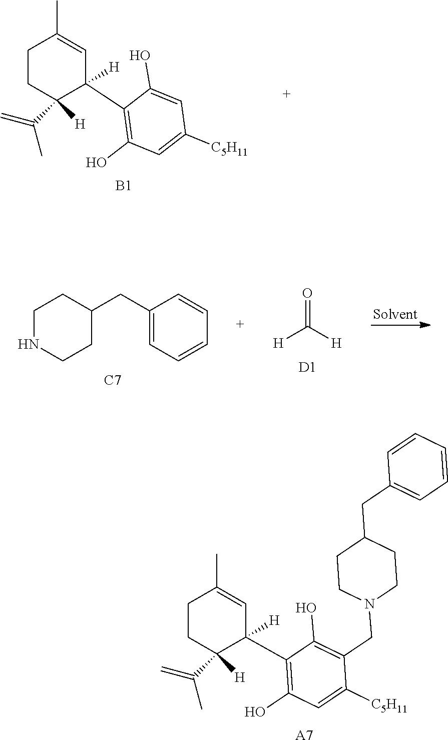

The present invention relates to the synthesis of a compound of formula A8 namely 2′-{(4-methylpiperazin-1-yl)methyl}-cannabidiol by the coupling of fragment B1 namely cannabidiol and fragment C8 namely 1-methylpiperazine in the presence of fragment D1 in a solvent.

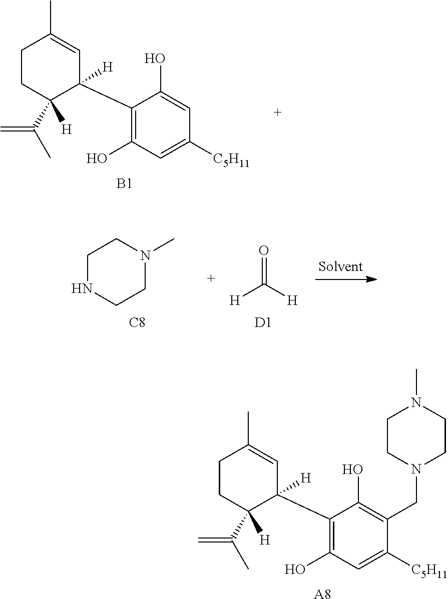

The present invention relates to the synthesis of a compound of formula A9 namely 2′-{(4-ethylpiperazin-1-yl)methyl}-cannabidiol by the coupling of fragment B1 namely cannabidiol and fragment C9 namely 1-ethylpiperazine in the presence of fragment D1 in a solvent.

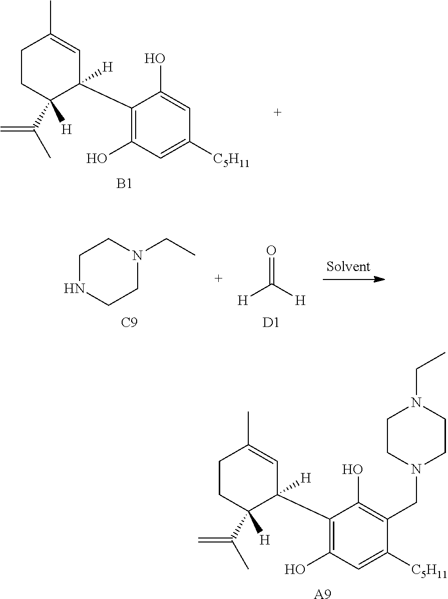

The present invention relates to the synthesis of a compound of formula A10 namely 2′-[{4-(2-hydroxyethyl)piperazin-1-yl}methyl]-cannabidiol by the coupling of fragment B1 namely cannabidiol and fragment C10 namely 2-(piperazin-1-yl)ethan-1-ol in the presence of fragment D1 in a solvent.

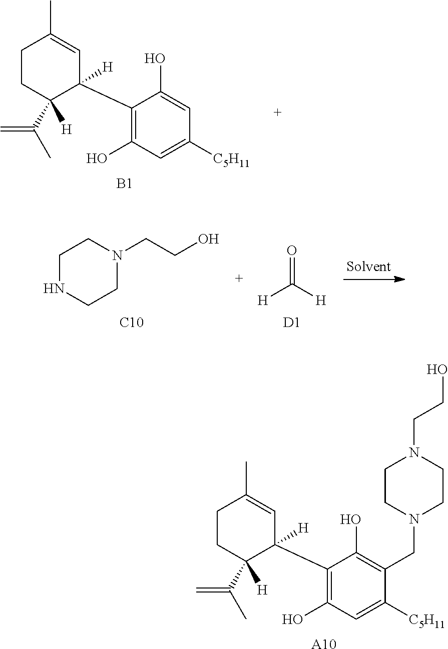

The present invention relates to the synthesis of a compound of formula A11 namely 2′-[{4-(piperazin-1-yl)ethan-1-one}methyl]-cannabidiol by the coupling of fragment B1 namely cannabidiol and fragment C11 namely 1-(piperazin-1-yl)ethan-1-one in the presence of fragment D1 in a solvent.

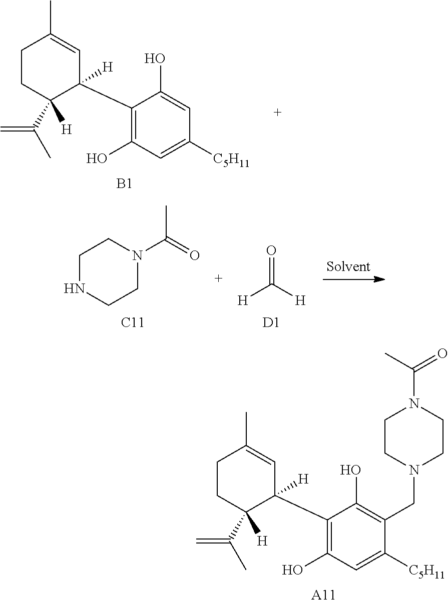

The present invention relates to the synthesis of a compound of formula A12 namely 2′-{(4-phenylpiperazin-1-yl)methyl}-cannabidiol by the coupling of fragment B1 namely cannabidiol and fragment C12 namely 1-phenylpiperazine in the presence of fragment D1 in a solvent.

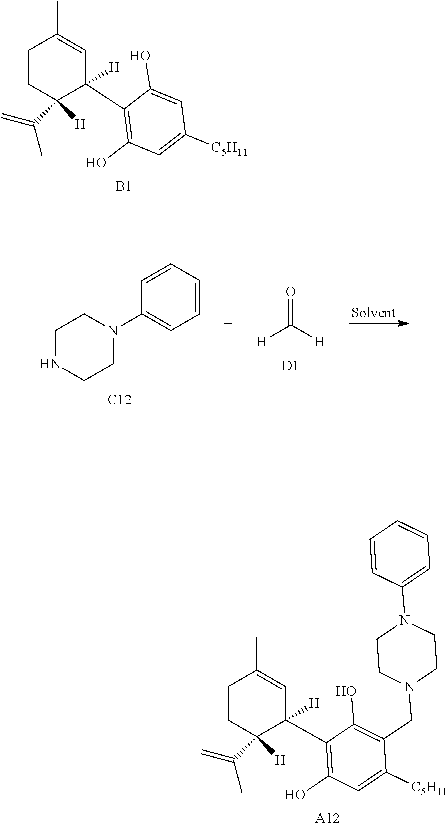

The present invention relates to the synthesis of a compound of formula A13 namely 2′-{(4-cyclopropylpiperazin-1-yl)methyl}-cannabidiol by the coupling of fragment B1 namely cannabidiol and fragment C13 namely 1-cyclopropylpiperazine in the presence of fragment D1 in a solvent.

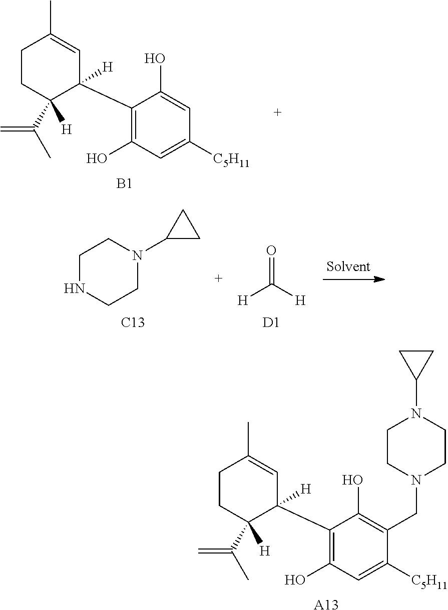

The present invention relates to the synthesis of a compound of formula A14 namely 2′-[{4-(4-chlorophenyl)piperazine}methyl]-cannabidiol by the coupling of fragment B1 namely cannabidiol and fragment C14 namely 1-(4-chlorophenyl) piperazine in the presence of fragment D1 in a solvent.

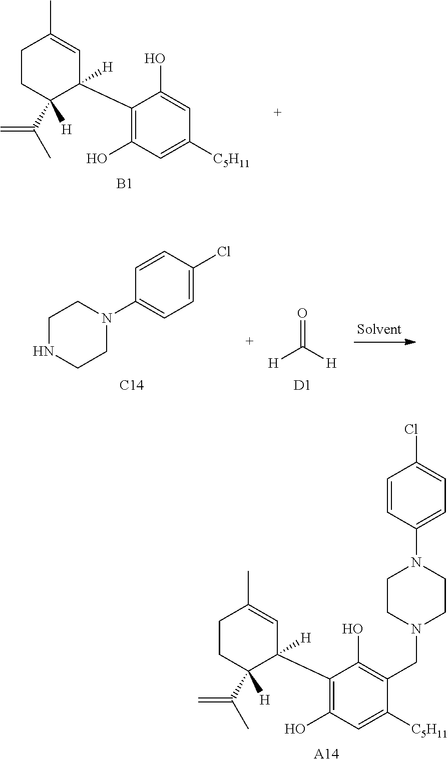

The present invention relates to the synthesis of a compound of formula A15 namely 2′-[{4-(pyrimidin-2-yl)piperazin-1-yl}methyl]-cannabidiol by the coupling of fragment B1 namely cannabidiol and fragment C15 namely 2-(piperazin-1-yl) pyrimidine in the presence of fragment D1 in a solvent.

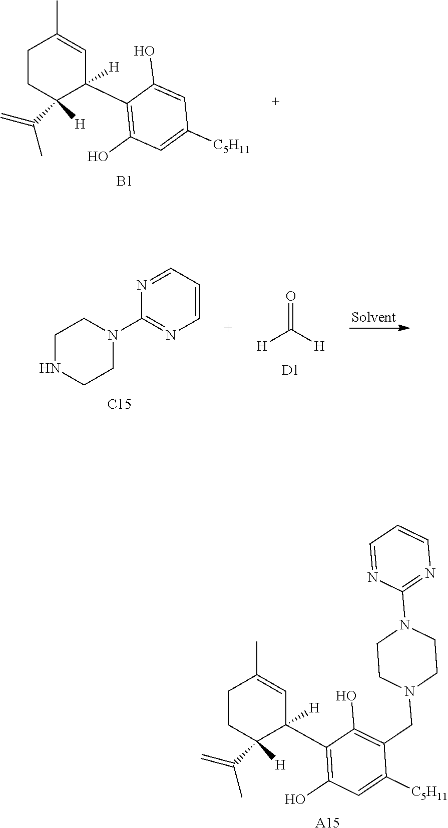

The present invention relates to the synthesis of a compound of formula A16 namely 2′-{(pyrazin-2-ylamino)methyl}-cannabidiol by the coupling of fragment B1 namely cannabidiol and fragment C16 namely 2-(piperazin-1-yl) pyrazine in the presence of fragment D1 in a solvent.

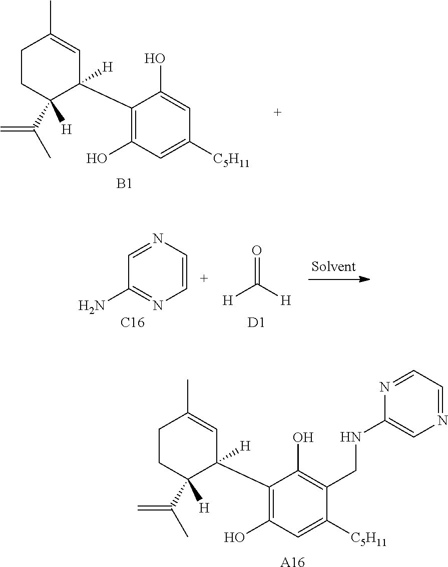

### Material and Method Used in Experiments

All the product mixtures were analyzed by thin layer chromatography. All novel analogues of cannabidiol of Formula A analyzed by charring regent such as anisaldehyde solution, dragendroff's solution and ninhydrin's solution. All the reactions were performed under inert atmosphere wherever required. NMR spectra (¹HNMR, ¹³C, DEPT) were recorded in 400 MHz spectrometer using CDCl₃ and CD₃OD solvent. ES1-MS and HRMS spectra were recorded on LC-MS/MS and HRMS-6540-UHD machines. Optical rotations were measured on a Perkin Elmer polarimeter. Column chromatography was carried out with silica gel (60-120, 100-200 and 230-400 mesh).

Although the subject matter has been described in considerable detail with reference to certain examples and implementations thereof, other implementations are possible.

### EXAMPLES

It should be understood that the disclosed examples are merely exemplary of the invention, which may be embodied in various forms. Therefore, the details disclosed herein are not to be interpreted as limiting but merely as the basis for the claims and as a basis for teaching one skilled in the art how to make or use the invention.

Following examples are given by way of illustration and therefore should not be construed to limit the scope of the invention.

### Example 1: Synthesis of 2′-(morpholin-1-ylmethyl)-cannabidiol (A1) and 2′,4′-{bis-(morpholin-1-ylmethyl)}-cannabidiol (A1′)

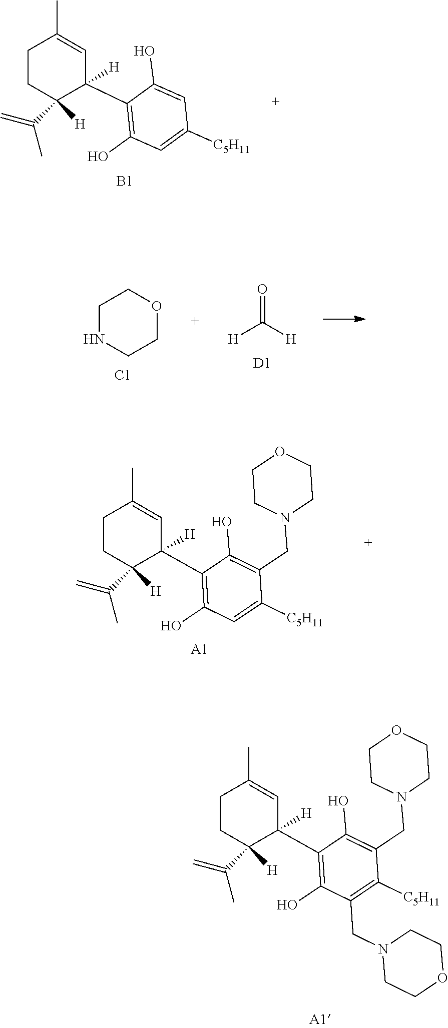

To a methanol solution of cannabidiol (B1, 150 mg, 0.477 mmol), formaldehyde solution (D, 37%, 154.5 μl) was added, followed by morpholine (C1, 124.4 mg, 1.431 mmol). For 15 hours, the reaction mixture was stirred at room temperature (rt). TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-(morpholin-1-ylmethyl)-cannabidiol (A1 or CS-16, 120 mg, 60.9%) and 2′,4′-{bis-(morpholin-1-ylmethyl)}-cannabidiol (A1′ or CS-84, 70 mg, 28.6%), the compounds were purified using column chromatography by eluted with ethyl acetate and hexane {R_{f}~0.7, 0.3 (10:90, Ethyl acetate:Hexane)}. ¹H NMR, ¹³C NMR, DEPT, and HRMS were used to characterize these derivatives. ¹H NMR (400 MHz, CDCl₃) δ 11.22 (bs, 1H, OH), 6.17 (s, 1H), 5.97 (s, 1H), 5.62 (s, 1H), 4.34 (d, J=23.5 Hz, 2H), 4.03 (d, J=8.4 Hz, 1H), 3.73-3.52 (m, 6H), 2.80-2.31 (m, 7H), 2.25-2.05 (m, 2H), 1.78 (s, 5H), 1.70 (s, 3H), 1.47-1.42 (m, 2H), 1.32-1.26 (m, 4H), 0.90-0.86 (m, 3H); ¹³C {¹H}NMR (101 MHz, CDCl₃) δ 156.7, 155.2, 148.1, 140.3, 139.4, 124.7, 114.7, 110.6, 109.9, 108.6, 66.8, 56.5, 52.4, 46.9, 35.6, 33.4, 31.7, 30.9, 30.4, 27.9, 23.8, 22.6, 19.1, 14.1; [α]_{D}²⁰=−142 (c=1.0, MeOH); HRMS (ESI-TOF) m/z: calcd for C₂₆H₄₀NO₃, 414.3008 [M+H]; found, 414.2999. ¹H NMR (400 MHz, CDCl₃) δ 11.01 (bs, 2H, OH), 5.37 (s, 1H), 4.36 (d, J=26.6 Hz, 2H), 4.03 (d, J=10.7 Hz, 1H), 3.89-3.47 (m, 12H), 3.22-3.11 (m, 1H), 2.91-2.31 (m, 10H), 2.27-1.99 (m, 2H), 1.81-1.75 (m, 2H), 1.71 (s, 3H), 1.59 (s, 3H), 1.39-1.35 (m, 6H), 0.93 (t, J=6.5 Hz, 3H); ¹³C {¹H}NMR (101 MHz, CDCl₃) δ 156.8, 150.5, 137.4, 131.5, 126.2, 116.6, 108.8, 66.7, 57.0, 52.5, 44.5, 36.5, 32.1, 30.8, 30.7, 29.7, 28.9, 23.6, 22.5, 18.9, 14.1; [α]_{D}²⁰=−96.6 (c=1.0, MeOH); HRMS (ESI-TOF) m/z: calcd for C₃₁H₄₉N₂O₄, 513.3692 [M+H]; found, 513.3689.

### Example 2: Synthesis of 2′-(piperidin-1-ylmethyl)-cannabidiol (A2)

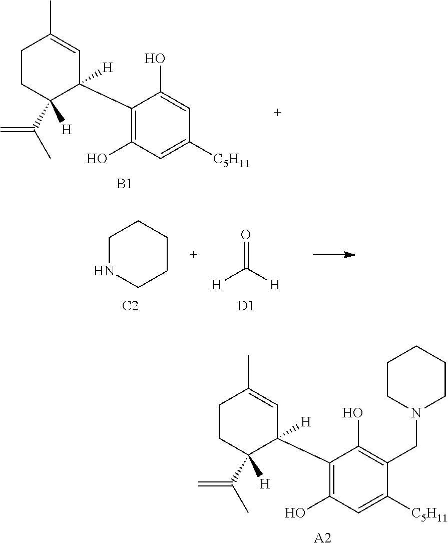

To a methanol solution of cannabidiol (B1, 120 mg, 0.382 mmol), formaldehyde solution (D1, 37%, 68.1 μl) was added, followed by piperidine (C2, 38.9 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-(piperidin-1-ylmethyl)-cannabidiol (A2 or CS-71, 102 mg, 64.9%), the compound was purified using column chromatography by eluted with ethyl acetate and hexane {R_{f}~0.6 (10:90, Ethyl acetate:Hexane)}. ¹H NMR, ¹³C NMR, DEPT, and HRMS were used to characterize these derivatives. ¹H NMR (400 MHz, CDCl₃) δ 6.13 (s, 1H), 5.62 (s, 1H), 4.39 (d, J=42.1 Hz, 2H), 4.03 (d, J=8.3 Hz, 1H), 3.56 (dd, J=51.2, 13.7 Hz, 2H), 3.26-2.52 (m, 2H), 2.49-2.38 (m, 3H), 2.35-1.97 (m, 4H), 1.81-1.77 (m, 5H), 1.71 (s, 3H), 1.68-1.39 (m, 8H), 1.34-1.28 (m, 4H), 0.88 (t, J=6.9 Hz, 3H); ¹³C {¹H}NMR (101 MHz, CDCl₃) δ 157.4, 154.9, 148.0, 139.9, 139.1, 124.9, 114.6, 110.7, 110.6, 108.1, 56.9, 53.4, 46.9, 35.7, 33.4, 31.7, 30.8, 30.4, 28.0, 25.9, 24.2, 23.7, 22.6, 19.1, 14.1; [α]_{D}²⁰=−118 (c=1.0, MeOH); HRMS (ESI-TOF) m/z: calcd for C₂₇H₄₂NO₂, 412.3216 [M+H]; found, 412.3216.

### Example 3: Synthesis of 2′-{(4-isopropylpiperidin-1-yl)methyl}-cannabidiol (A3)

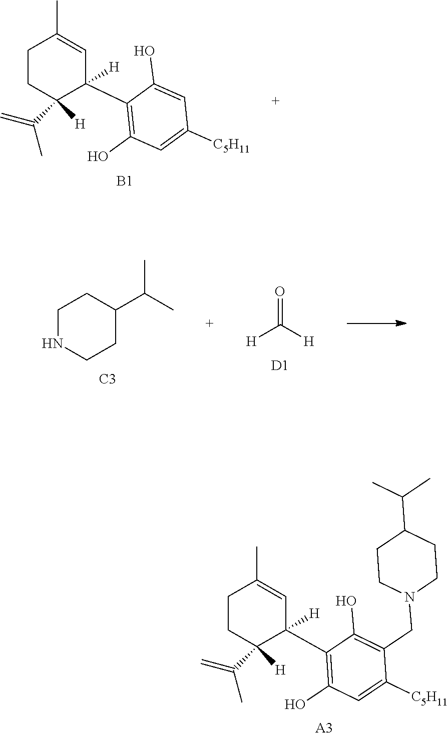

To a methanol solution of cannabidiol (B1, 120 mg, 0.382 mmol), formaldehyde solution (D1, 37%, 68.1 μl) was added, followed by isopropyl piperidine (C3, 58.2 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(4-isopropylpiperidin-1-yl)methyl}-cannabidiol (A3 or CS-86, 50 mg, 28.9%), the compound was purified using column chromatography by eluted with ethyl acetate and hexane {R_{f}~0.8 (10:90, Ethyl acetate:Hexane}. ¹H NMR, ¹³C NMR, DEPT, and HRMS were used to characterize these derivatives. ¹H NMR (400 MHz, CDCl₃) δ 6.14 (s, 1H), 5.93 (bs, 1H, OH), 5.62 (s, 1H), 4.40 (d, J=43.0 Hz, 2H), 4.03 (d, J=8.2 Hz, 1H), 3.57 (dd, J=54.8, 13.7 Hz, 2H), 3.03-2.87 (m, 2H), 2.48-2.34 (m, 3H), 2.25-1.97 (m, 4H), 1.81-1.78 (m, 5H), 1.71 (s, 3H), 1.68-1.59 (m, 2H), 1.49-1.40 (m, 3H), 1.33-1.19 (m, 6H), 1.09-1.02 (m, 1H), 0.90-0.87 (m, 9H); ¹³C {¹H}NMR (101 MHz, CDCl₃) δ 157.4, 154.8, 148.0, 139.9, 139.1, 124.9, 114.6, 110.9, 110.6, 108.1, 56.6, 54.0, 52.2, 46.9, 42.3, 35.7, 33.4, 32.3, 31.7, 30.7, 30.4, 29.4, 29.1, 28.0, 23.7, 22.6, 19.8, 19.7, 19.1, 14.1; [α]_{D}²⁰=−84.6 (c=1.0, MeOH); HRMS (ESI-TOF) m/z: calcd for C₃₀H₄₈NO₂, 454.3685 [M+H]; found, 454.3680.

### Example 4: Synthesis of 2′-{(4-hydroxypiperidin-1-yl)methyl}-cannabidiol (A4)

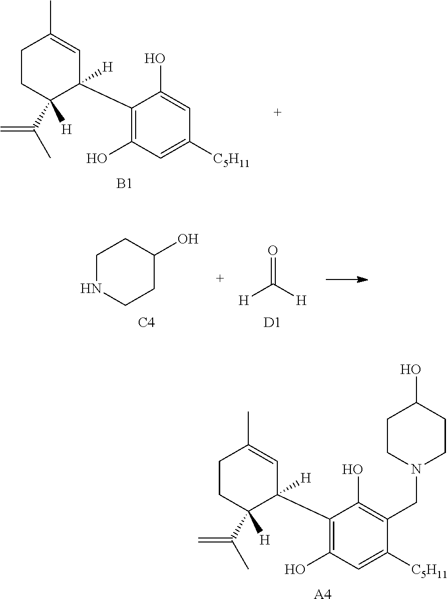

To a methanol solution of cannabidiol (B1, 120 mg, 0.382 mmol), formaldehyde solution (D1, 37%, 68.1 μl) was added, followed by piperidin-4-ol (C4, 46.2 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(4-hydroxypiperidin-1-yl)methyl}-cannabidiol (A4 or CS-73, 98 mg, 60.1%), the compound was purified using column chromatography by eluted with ethyl acetate and hexane {R_{f}~0.5 (20:80, Ethyl acetate:Hexane)}. ¹H NMR, ¹³C NMR, DEPT, and HRMS were used to characterize these derivatives. ¹H NMR (400 MHz, CDCl₃) δ 6.15 (s, 1H), 5.95 (bs, 1H, OH), 5.62 (s, 1H), 4.38 (d, J=42.8 Hz, 2H), 4.02 (d, J=8.5 Hz, 1H), 3.92-3.42 (m, 3H), 2.98-2.63 (m, 2H), 2.48-2.35 (m, 3H), 2.35-2.01 (m, 4H), 1.92-1.84 (m, 2H), 1.81-1.75 (m, 5H), 1.71 (s, 3H), 1.65-1.55 (m, 2H), 1.48-1.40 (m, 2H), 1.31-1.25 (m, 4H), 0.88 (t, J=6.9 Hz, 3H); ¹³C {¹H}NMR (101 MHz, CDCl₃) δ 157.2, 154.9, 148.0, 139.9, 139.3, 124.8, 114.7, 110.6, 110.5, 108.3, 56.2, 49.4, 46.9, 35.7, 34.23, 33.4, 31.7, 30.8, 30.4, 28.0, 23.8, 22.6, 19.1, 14.1; [α]_{D}²⁰=−96 (c=1.0, MeOH); HRMS (ESI-TOF) m/z: calcd for C₂₇H₄₂NO₃, 428.3165 [M+H]; found, 428.3160.

### Example 5: Synthesis of Ring Annulated Analogues of 2′-{(4-phenylpiperidin-1-yl)methyl}-cannabidiol (A5)

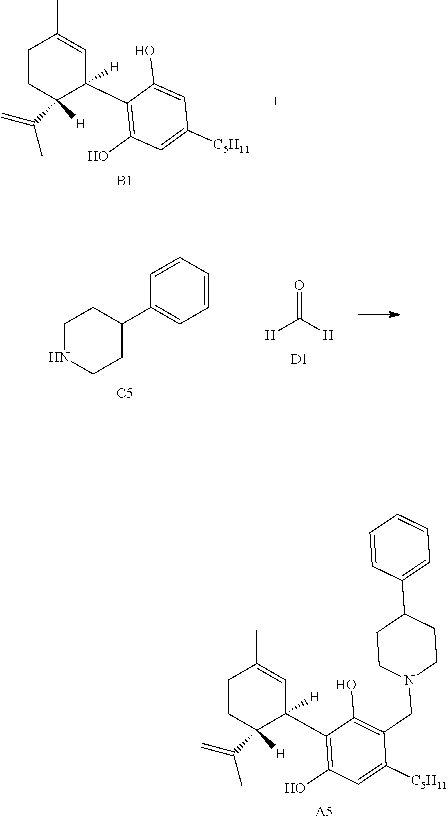

To a methanol solution of cannabidiol (B1, 120 mg, 0.382 mmol), formaldehyde solution (D1, 37%, 68.1 μl) was added, followed by 4-phenylpiperidine (C5, 73.8 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(4-phenylpiperidin-1-yl)methyl}-cannabidiol (A5 or CS-74, 140 mg, 75.2%), the compound was purified using column chromatography by eluted with ethyl acetate and hexane {R_{f}~0.7 (10:90, Ethyl acetate:Hexane)}. ¹H NMR, ¹³C NMR, DEPT, and HRMS were used to characterize these derivatives. ¹H NMR (400 MHz, CDCl₃) δ 7.26-7.13 (m, 5H), 6.09 (s, 1H), 5.88 (bs, 1H, OH), 5.56 (s, 1H), 5.05 (bs, 1H, OH), 4.32 (d, J=35.9 Hz, 2H), 3.98 (d, J=7.2 Hz, 1H), 3.57 (dd, J=51.7, 13.6 Hz, 2H), 3.08-2.91 (m, 2H), 2.52-2.46 (m, 1H), 2.42-2.30 (m, 3H), 2.20-1.94 (m, 4H), 1.85-1.77 (m, 2H), 1.71 (s, 5H), 1.65 (s, 3H), 1.60-1.52 (m, 2H), 1.42-1.35 (m, 2H), 1.25-1.24 (m, 4H), 0.82 (t, J=6.5 Hz, 3H); ¹³C {¹H}NMR (101 MHz, CDCl₃) δ 157.3, 154.9, 148.0, 145.7, 139.9, 139.3, 128.6, 126.8, 126.4, 124.9, 114.7, 110.7, 108.3, 56.6, 54.1, 52.2, 46.9, 42.4, 39.8, 35.7, 33.5, 33.3, 31.8, 30.8, 30.4, 28.0, 23.8, 22.6, 19.2, 14.1; [α]_{D}²⁰=−46.3 (c=1.0, MeOH); HRMS (ESI-TOF) m/z: calcd for C₃₃H₄₆NO₂, 488.3529 [M+H]; found, 488.3527.

### Example 6: Synthesis of 2′-[{4-(trifluoromethyl)piperidin-1-yl}methyl]-cannabidiol (A6)

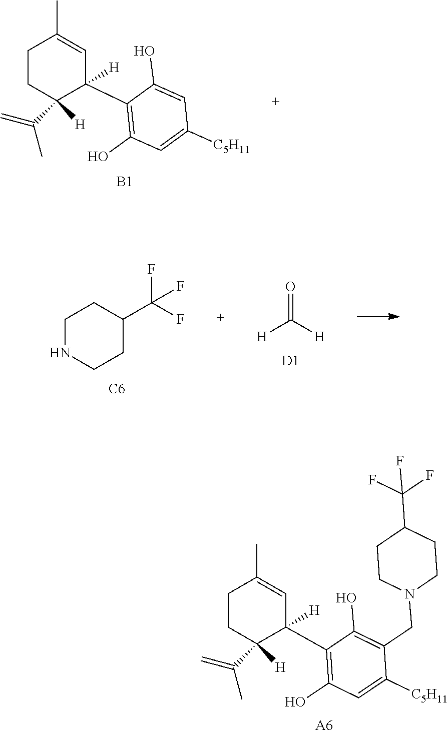

To a methanol solution of cannabidiol (B1, 120 mg, 0.382 mmol), formaldehyde solution (D1, 37%, 68.1 μl) was added, followed by 4-(trifluoromethyl) piperidine (C6, 70.1 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-[{4-(trifluoromethyl)piperidin-1-yl}methyl]-cannabidiol (A6 or CS-75, 145 mg, 79.2%), the compound was purified using column chromatography by eluted with ethyl acetate and hexane {R_{f}~0.5 (10:90, Ethyl acetate:Hexane)}. ¹H NMR, ¹³C NMR, DEPT, and HRMS were used to characterize these derivatives. ¹H NMR (400 MHz, CDCl₃) δ 11.03 (bs, 1H, OH), 6.16 (s, 1H), 5.97 (bs, 1H, OH), 5.62 (s, 1H), 4.38 (d, J=41.9 Hz, 2H), 4.03 (d, J=8.3 Hz, 1H), 3.69-3.53 (m, 2H), 3.04 (dd, J=56.7, 11.3 Hz, 2H), 2.47-2.36 (m, 3H), 2.26-2.05 (m, 4H), 1.94-1.81 (m, 4H), 1.80-1.76 (m, 5H), 1.71 (s, 3H), 1.60-1.53 (m, 1H), 1.48-1.41 (m, 2H), 1.32-1.26 (m, 4H), 0.89 (t, J=6.9 Hz, 3H); ¹³C {¹H}NMR (101 MHz, CDCl₃) δ 156.9, 155.1, 148.0, 140.0, 139.4, 131.4, 128.6, 125.8, 124.7, 114.8, 110.5, 110.1, 108.5, 56.3, 52.1, 50.4, 46.9, 40.6, 40.4, 40.2, 39.8, 35.7, 33.4, 31.7, 30.8, 30.4, 27.9, 24.7, 24.5, 23.7, 22.6, 19.1, 14.1; [α]_{D}²⁰=−75 (c=1.0, MeOH); HRMS (ESI-TOF) m/z: calcd for C₂₈H₄₁F₃NO₂, 480.3089 [M+H]; found, 480.3082.

### Example 7: Synthesis of 2′-{(4-benzylpiperidin-1-yl)methyl}-cannabidiol (A7)

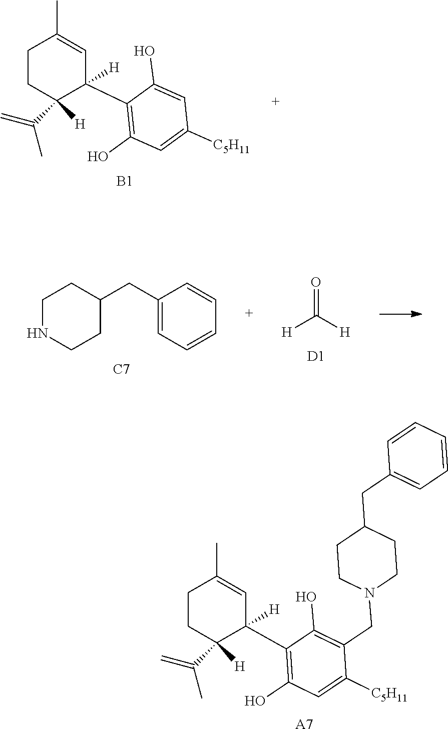

To a methanol solution of cannabidiol (B1, 120 mg, 0.382 mmol), formaldehyde solution (D1, 37%, 68.1 μl) was added, followed by 4-benzylpiperidine (C7, 80.1 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(4-benzylpiperidin-1-yl)methyl}-cannabidiol (A7 or CS-76, 140 mg, 73.2%), the compound was purified using column chromatography by eluted with ethyl acetate and hexane {R_{f}~0.7 (10:90, Ethyl acetate:Hexane)}. ¹H NMR, ¹³C NMR, DEPT, and HRMS were used to characterize these derivatives. ¹H NMR (400 MHz, CDCl₃) δ 7.23-7.05 (m, 5H), 6.06 (s, 1H), 5.86 (bs, 1H, OH), 5.55 (s, 1H), 4.32 (d, J=40.8 Hz, 2H), 3.96 (d, J=7.2 Hz, 1H), 3.57-3.40 (m, 2H), 2.89-2.76 (m, 2H), 2.52-2.45 (m, 2H), 2.39-2.26 (m, 3H), 2.23-1.88 (m, 4H), 1.81-1.70 (m, 6H), 1.64-1.42 (m, 7H), 1.39-1.32 (m, 2H), 1.22-1.18 (m, 4H), 0.80 (t, J=6.8 Hz, 3H); ¹³C {¹H}NMR (101 MHz, CDCl₃) δ 157.3, 154.9, 148.0, 140.4, 139.9, 139.2, 129.1, 128.3, 125.9, 124.9, 114.6, 110.7, 110.6, 108.2, 56.6, 53.6, 51.9, 46.9, 43.0, 37.8, 35.7, 33.4, 32.1, 31.7, 30.7, 30.4, 28.0, 23.8, 22.6, 19.2, 14.1; [α]_{D}²⁰=−82.6 (c=1.0, MeOH); HRMS (ESI-TOF) m/z: calcd for C₃₄H₄₈NO₂, 502.3685 [M+H]; found, 502.3673.

### Example 8: Synthesis of 2′-{(4-methylpiperazin-1-yl)methyl}-cannabidiol (A8)

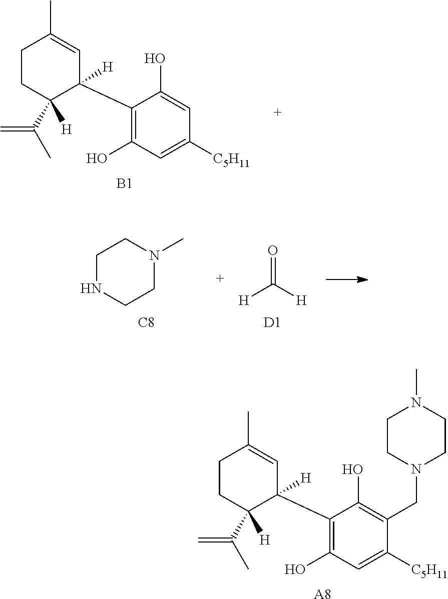

To a methanol solution of cannabidiol (B1, 120 mg, 0.382 mmol), formaldehyde solution (D1, 37%, 68.1 μl) was added, followed by 1-methylpiperazine (C8, 45.8 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(4-methylpiperazin-1-yl)methyl}-cannabidiol (A8 or CS-77, 140 mg, 86.4%), the compound was purified using column chromatography by eluted with ethyl acetate and hexane {R_{f}~0.3 (100:0, Ethyl acetate:Hexane)}. ¹H NMR, ¹³C NMR, DEPT, and HRMS were used to characterize these derivatives. ¹H NMR (400 MHz, CDCl₃) δ 6.14 (s, 1H), 5.98 (bs, 1H, OH), 5.61 (s, 1H), 4.36 (d, J=31.0 Hz, 2H), 4.02 (d, J=8.9 Hz, 1H), 3.68-3.51 (m, 2H), 3.40-2.51 (m, 6H), 2.44-2.37 (m, 3H), 2.30 (s, 3H), 2.24-2.03 (m, 4H), 1.80-1.76 (m, 5H), 1.70 (s, 3H), 1.46-1.40 (m, 2H), 1.32-1.25 (m, 4H), 0.87 (t, J=6.9 Hz, 3H); ¹³C {¹H}NMR (101 MHz, CDCl₃) δ 156.9, 155.0, 148.0, 140.1, 139.3, 124.8, 114.7, 110.6, 110.3, 108.4, 56.1, 54.9, 52.1, 46.8, 45.9, 35.7, 33.4, 31.7, 30.8, 30.4, 28.0, 23.7, 22.6, 19.1, 14.1; [α]_{D}²⁰=−85.6 (c=1.0, MeOH); HRMS (ESI-TOF) m/z: calcd for C₂₇H₄₃N₂O₂, 427.3325 [M+H]; found, 427.3324.

### Example 9: Synthesis of 2′-{(4-ethylpiperazin-1-yl)methyl}-cannabidiol (A9)

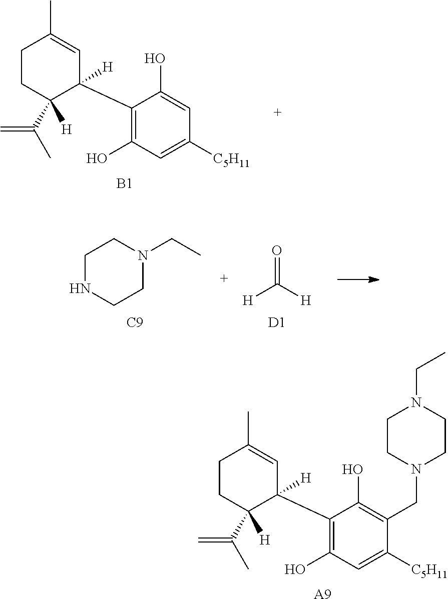

To a methanol solution of cannabidiol (B1, 120 mg, 0.382 mmol), formaldehyde solution (D1, 37%, 68.1 μl) was added, followed by 1-ethylpiperazine (C9, 52.2 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(4-ethylpiperazin-1-yl)methyl}-cannabidiol (A9 or CS-78, 150 mg, 89.2%), the compound was purified using column chromatography by eluted with ethyl acetate and hexane {R_{f}~0.4 (50:50, Ethyl acetate:Hexane)}. ¹H NMR, ¹³C NMR, DEPT, and HRMS were used to characterize these derivatives. ¹H NMR (400 MHz, CDCl₃) δ 6.08 (s, 1H), 5.89 (bs, 1H, OH), 5.54 (s, 1H), 4.30 (d, J=32.9 Hz, 2H), 3.95 (d, J=8.7 Hz, 1H), 3.54 (dd, J=52.3, 13.6 Hz, 2H), 3.33-2.59 (m, 4H), 2.48-2.24 (m, 7H), 2.18-1.94 (m, 4H), 1.73-1.68 (m, 5H), 1.63 (s, 3H), 1.42-1.34 (m, 2H), 1.26-1.18 (m, 4H), 1.03 (t, J=7.2 Hz, 3H), 0.81 (t, J=6.9 Hz, 3H); ¹³C {¹H}NMR (101 MHz, CDCl₃) δ 156.9, 155.0, 148.0, 140.1, 139.3, 124.8, 114.6, 110.6, 110.3, 108.4, 56.2, 52.6, 52.2, 49.4, 46.8, 35.7, 33.4, 31.7, 30.8, 30.4, 28.0, 23.7, 22.6, 19.1, 14.1, 12.0; [α]_{D}²⁰=−93.3 (c=1.0, MeOH); HRMS (ESI-TOF) m/z: calcd for C₂₈H₄₅N₂O₂, 441.3481 [M+H]; found, 441.3481.

### Example 10: Synthesis of 2′-[{4-(2-hydroxyethyl)piperazin-1-yl}methyl]-cannabidiol (A10)

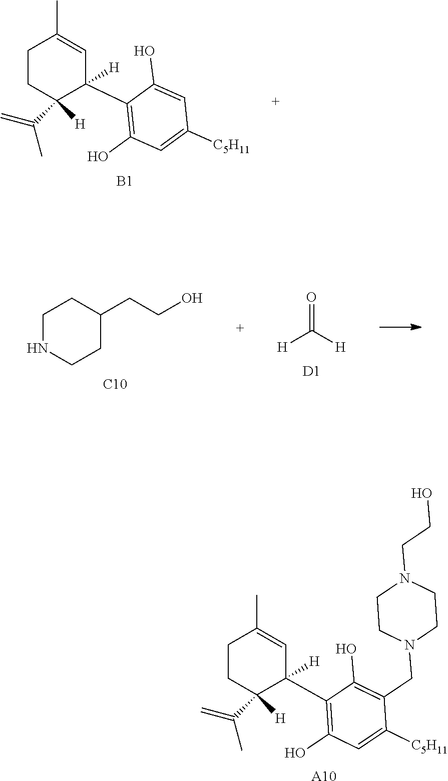

To a methanol solution of cannabidiol (B1, 120 mg, 0.382 mmol), formaldehyde solution (D1, 37%, 68.1 μl) was added, followed by 2-(piperazin-1-yl) ethan-1-ol (C10, 59.5 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-[{4-(2-hydroxyethyl)piperazin-1-yl}methyl]-cannabidiol (A10 or CS-85, 90 mg, 51.7%), the compound was purified using column chromatography by eluted with methanol and chloroform{R_{f}~0.5 (10:90, Methanol:Chloroform)}. ¹H NMR, ¹³C NMR, DEPT, and HRMS were used to characterize these derivatives. ¹H NMR (400 MHz, CDCl₃) δ 6.14 (s, 1H), 5.94 (s, 1H), 5.59 (s, 1H), 4.35 (d, J=28.9 Hz, 2H), 4.00 (d, J=8.7 Hz, 1H), 3.68-3.52 (m, 4H), 3.44-2.62 (m, 4H), 2.57-2.55 (m, 3H), 2.45-2.35 (m, 4H), 2.29-2.03 (m, 4H), 1.76 (s, 5H), 1.69 (s, 3H), 1.48-1.39 (m, 2H), 1.33-1.25 (m, 4H), 0.87 (t, J=6.8 Hz, 3H); ¹³C NMR (101 MHz, CDCl₃) δ 156.9, 155.0, 148.0, 140.1, 139.3, 124.7, 114.7, 110.6, 110.2, 108.5, 59.2, 57.8, 56.1, 52.7, 51.9, 46.8, 35.7, 33.4, 31.7, 30.8, 30.4, 28.0, 23.7, 22.6, 19.1, 14.1; [α]_{D}²⁰=−91 (c=1.0, MeOH); HRMS (ESI-TOF) m/z: calcd for C₂₈H₄₅N₂O₃, 457.3430 [M+H]; found, 457.3424.

### Example 11: Synthesis of 2′-[{4-(piperazin-1-yl)ethan-1-one}methyl]-cannabidiol (A11)

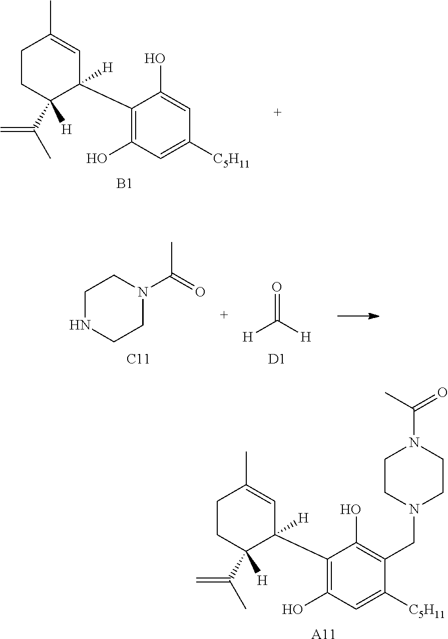

To a methanol solution of cannabidiol (B1, 120 mg, 0.382 mmol), formaldehyde solution (D1, 37%, 68.1 μl) was added, followed by 1-(piperazin-1-yl) ethan-1-one (C11, 58.6 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-[{4-(piperazin-1-yl)ethan-1-one}methyl]-cannabidiol (A11 or CS-79, 130 mg, 75%), the compound was purified using column chromatography by eluted with ethyl acetate and hexane {R_{f}~0.5 (70:30, Ethyl acetate:Hexane)}. ¹H NMR, ¹³C NMR, DEPT, and HRMS were used to characterize these derivatives. ¹H NMR (400 MHz, CDCl₃) δ 6.11 (s, 1H), 5.93 (bs, 1H, OH), 5.54 (s, 1H), 4.30 (d, J=35.2 Hz, 2H), 3.95 (d, J=8.4 Hz, 1H), 3.65-3.39 (m, 4H), 3.32-2.08 (m, 11H), 2.03 (s, 3H), 1.71 (s, 5H), 1.63 (s, 3H), 1.39-1.33 (m, 2H), 1.24-1.18 (m, 4H), 0.81 (t, J=6.7 Hz, 3H); ¹³C {¹H}NMR (101 MHz, CDCl₃) δ 168.9, 156.6, 155.3, 148.1, 140.2, 139.5, 124.6, 114.8, 110.6, 109.8, 108.8, 56.1, 52.1, 51.7, 46.8, 46.1, 41.2, 35.7, 33.4, 31.7, 30.8, 30.4, 27.9, 23.7, 22.5, 21.3, 19.1, 14.1; [α]_{D}²⁰=−68.6 (c=1.0, MeOH); HRMS (ESI-TOF) m/z: calcd for C₂₈H₄₃N₂O₃, 455.3274 [M+H]; found, 455.3272.

### Example 12: Synthesis of 2′-{(4-phenylpiperazin-1-yl)methyl}-cannabidiol (A12)

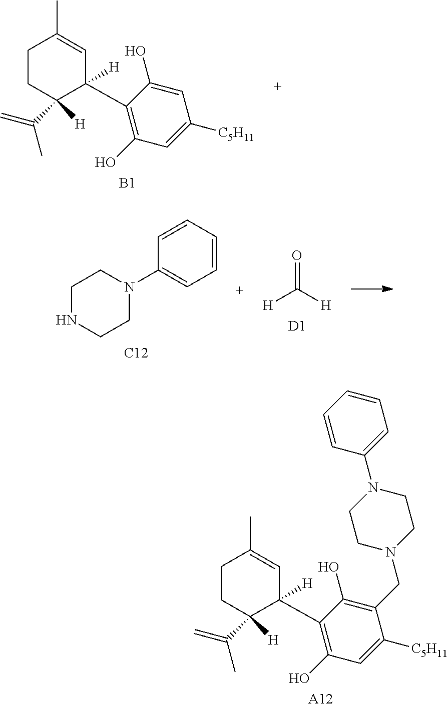

To a methanol solution of cannabidiol (B1, 120 mg, 0.382 mmol), formaldehyde solution (D1, 37%, 68.1 μl) was added, followed by 1-phenylpiperazine (C12, 74.1 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(4-phenylpiperazin-1-yl)methyl}-cannabidiol (A12 or CS-80, 130 mg, 69.8%), the compound was purified using column chromatography by eluted with ethyl acetate and hexane {R_{f}~0.7 (10:90, Ethyl acetate:Hexane)}. ¹H NMR, ¹³C NMR, DEPT, and HRMS were used to characterize these derivatives. ¹H NMR (400 MHz, CDCl₃) δ 11.15 (bs, 1H, OH), 7.23-7.18 (m, 2H), 6.91-6.77 (m, 3H), 6.11 (s, 1H), 5.90 (s, 1H), 5.55 (s, 1H), 4.31 (d, J=36.3 Hz, 2H), 3.96 (d, J=8.4 Hz, 1H), 3.60 (dd, J=58.9, 13.5 Hz, 2H), 3.48-3.41 (m, 8H), 2.39-2.23 (m, 3H), 2.20-1.98 (m, 2H), 1.73-1.67 (m, 5H), 1.62 (s, 3H), 1.44-1.36 (m, 2H), 1.28-1.20 (m, 4H), 0.82 (t, J=6.9 Hz, 3H); ¹³C {¹H}NMR (101 MHz, CDCl₃) δ 156.9, 155.1, 150.9, 148.1, 140.2, 139.4, 129.2, 124.7, 120.2, 116.3, 114.8, 110.7, 110.2, 108.6, 56.2, 52.0, 49.2, 46.9, 35.7, 33.5, 31.7, 30.9, 30.4, 28.0, 23.8, 22.6, 19.1, 14.1; [α]_{D}²⁰=−87 (c=1.0, MeOH); HRMS (ESI-TOF) m/z: calcd for C₃₂H₄₅N₂O₂, 489.3481 [M+H]; found, 489.3481.

### Example 13: Synthesis of 2′-{(4-cyclopropylpiperazin-1-yl)methyl}-cannabidiol (A13)

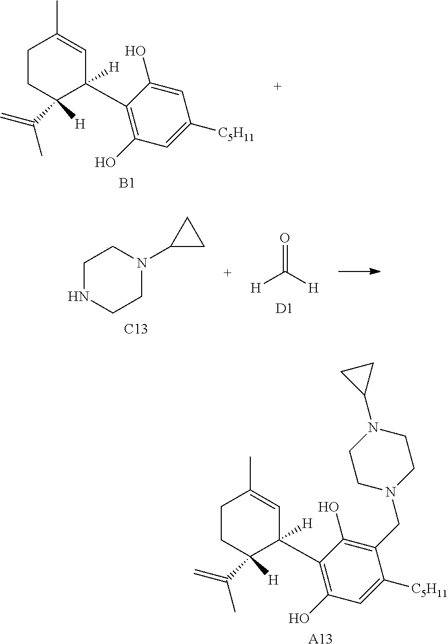

To a methanol solution of cannabidiol (B1, 120 mg, 0.382 mmol), formaldehyde solution (D1, 37%, 68.1 μl) was added, followed by 1-cyclopropylpiperazine (C13, 57.7 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(4-cyclopropylpiperazin-1-yl)methyl}-cannabidiol (A13 or CS-81, 130 mg, 75.5%), the compound was purified using column chromatography by eluted with ethyl acetate and hexane {R_{f}~0.5 (10:90, Ethyl acetate:Hexane)}. ¹H NMR, ¹³C NMR, DEPT, and HRMS were used to characterize these derivatives. ¹H NMR (400 MHz, CDCl₃) δ 6.15 (s, 1H), 5.95 (bs, 1H, OH), 5.62 (s, 1H), 4.38 (d, J=38.0 Hz, 2H), 4.04 (d, J=8.4 Hz, 1H), 3.60 (dd, J=57.8, 13.6 Hz, 2H), 3.09-2.48 (m, 6H), 2.43-2.38 (m, 3H), 2.22-2.06 (m, 2H), 1.80-1.75 (m, 5H), 1.72 (s, 3H), 1.65-1.60 (m, 1H), 1.48-1.40 (m, 2H), 1.33-1.25 (m, 6H), 0.87 (t, J=6.9 Hz, 3H), 0.48-0.39 (m, 4H); ¹³C NMR (101 MHz, CDCl₃) δ 157.0, 155.0, 148.0, 140.0, 139.3, 124.8, 114.6, 110.6, 110.4, 108.4, 56.2, 53.1, 52.1, 46.9, 38.3, 35.7, 33.4, 31.7, 30.8, 30.4, 28.0, 23.7, 22.5, 19.1, 14.1, 5.8; [α]_{D}²⁰=−101.6 (c=1.0, MeOH); HRMS (ESI-TOF) m/z: calcd for C₂₉H₄₅N₂O₂, 453.3481 [M+H]; found, 453.3483.

### Example 14: Synthesis of 2′-[{4-(4-chlorophenyl)piperazine}methyl]-cannabidiol (A14)

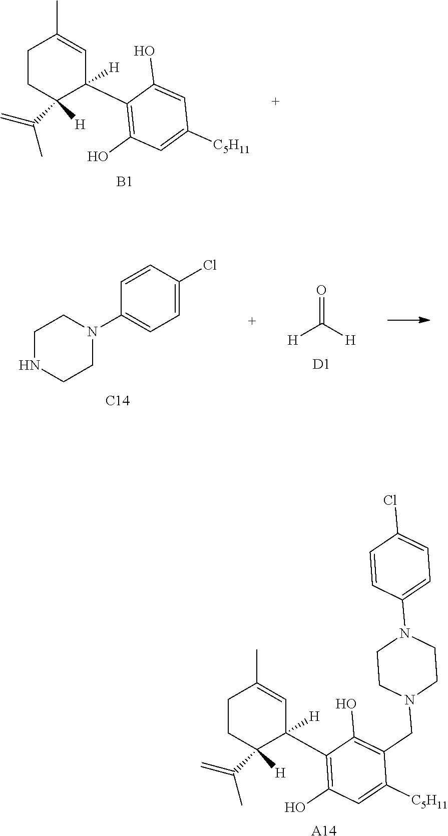

To a methanol solution of cannabidiol (B1, 120 mg, 0.382 mmol), formaldehyde solution (D1, 37%, 68.1 μl) was added, followed by 1-(4-chlorophenyl) piperazine (C14, 89.7 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-[{4-(4-chlorophenyl)piperazine}methyl]-cannabidiol (A14 or CS-82, 101 mg, 50.7%), the compound was purified using column chromatography by eluted with ethyl acetate and hexane {R_{f}~0.7 (10:90, Ethyl acetate:Hexane)}. ¹H NMR, ¹³C NMR, DEPT, and HRMS were used to characterize these derivatives. ¹H NMR (400 MHz, CDCl₃) δ 7.25-7.20 (m, 2H), 6.86-6.82 (m, 2H), 6.19 (s, 1H), 5.91 (bs, 1H, OH), 5.62 (s, 1H), 4.38 (d, J=33.9 Hz, 2H), 4.03 (d, J=9.2 Hz, 1H), 3.67 (dd, J=56.2, 13.5 Hz, 2H), 3.51-2.47 (m, 8H), 2.46-2.33 (m, 3H), 2.25-2.03 (m, 2H), 1.78 (s, 5H), 1.70 (s, 3H), 1.50-1.43 (m, 2H), 1.34-1.28 (m, 4H), 0.89 (t, J=7.0 Hz, 3H); ¹³C {¹H}NMR (101 MHz, CDCl₃) δ 156.8, 155.2, 149.5, 148.1, 140.2, 139.5, 129.1, 125.0, 124.7, 117.5, 114.8, 110.8, 110.3, 108.7, 56.2, 51.9, 49.2, 46.8, 35.7, 33.5, 31.7, 30.9, 30.4, 28.0, 23.8, 22.6, 19.2, 14.1; [α]_{D}²⁰=−98 (c=1.0, MeOH); HRMS (ESI-TOF) m/z: calcd for C₃₂H₄₄ClN₂O₂, 523.3091 [M+H]; found, 523.3090.

### Example 15: Synthesis of 2′-[{4-(pyrimidin-2-yl)piperazin-1-yl}methyl]-cannabidiol (A15)

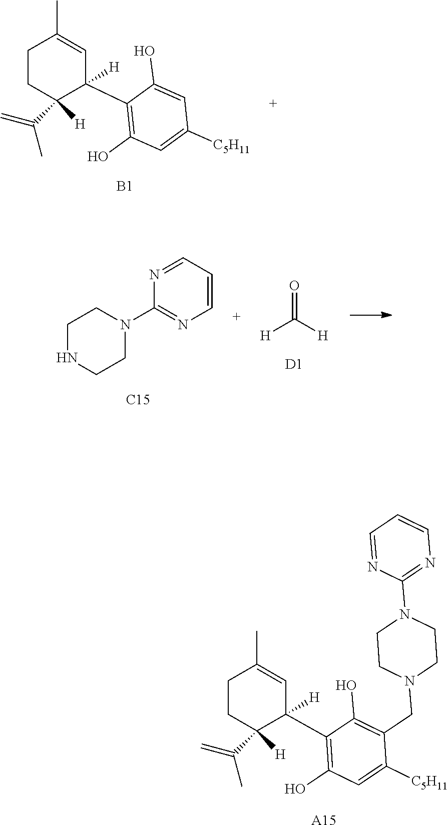

To a methanol solution of cannabidiol (B1, 120 mg, 0.382 mmol), formaldehyde solution (D1, 37%, 68.1 μl) was added, followed by 2-(piperazin-1-yl) pyrimidine (C15, 75.2 mg, 0.458 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-[{4-(pyrimidin-2-yl)piperazin-1-yl}methyl]-cannabidiol (A15 or CS-83, 105 mg, 56.1%), the compound was purified using column chromatography by eluted with ethyl acetate and hexane {R_{f}~0.6 (10:90, Ethyl acetate:Hexane)}. ¹H NMR, ¹³C NMR, DEPT, and HRMS were used to characterize these derivatives. ¹H NMR (400 MHz, CDCl₃) δ 10.53 (bs, 1H, OH), 7.98-7.79 (m, 3H), 6.29 (s, 1H), 6.04 (s, 1H), 5.45 (d, J=100.0 Hz, 2H), 4.52-4.16 (m, 5H), 2.63-2.52 (m, 2H), 2.39-2.04 (m, 3H), 1.81-1.77 (m, 5H), 1.67 (s, 3H), 1.62-1.55 (m, 2H), 1.39-1.35 (m, 4H), 0.91 (t, J=6.2 Hz, 3H); ¹³C {¹H}NMR (101 MHz, CDCl₃) δ 154.0, 152.2, 147.4, 139.8, 139.8, 138.7, 129.0, 128.4, 127.3, 124.4, 114.4, 111.1, 109.3, 109.0, 81.6, 55.4, 47.5, 46.8, 35.2, 31.7, 31.6, 30.5, 29.6, 28.1, 23.7, 22.5, 18.8, 14.0; [α]_{D}²⁰=−83 (c=1.0, MeOH); HRMS (ESI-TOF) m/z: calcd for C₃₀H₄₃N₄O₂, 491.3386 [M+H]; found, 491.3382.

### Example 16: Synthesis of 2′-{(pyrazin-2-ylamino)methyl}-cannabidiol (A16)

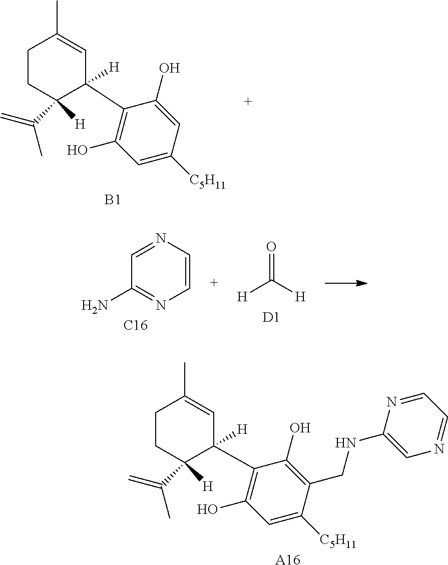

To a methanol solution of cannabidiol (B1, 120 mg, 0.382 mmol), formaldehyde solution (D1, 37%, 68.1 μl) was added, followed by pyrazin-2-amine (C16, 73 mg, 0.764 mmol). For 15 hours, the reaction mixture was stirred at room temperature. TLC was used to monitor the reaction mixture and check for reactant consumption. After the reaction was completed, the reaction mixture was extracted using water and ethyl acetate (2×200 volume). On a rota evaporator, the organic layer was collected and concentrated in vacuo. To obtain the 2′-{(pyrazin-2-ylamino)methyl}-cannabidiol (A16 or CS-70, 168 mg, 61%), the compound was purified using column chromatography by eluted with ethyl acetate and hexane {R_{f}~0.5 (30:70, Ethyl acetate:Hexane)}. ¹H NMR, ¹³C NMR, DEPT, and HRMS were used to characterize these derivatives. ¹H NMR (400 MHz, CDCl₃) δ 10.53 (bs, 1H, OH), 8.01-7.75 (m, 3H), 6.29 (s, 1H), 6.04 (bs, 1H, OH), 5.57 (s, 1H), 5.28 (bs, 1H, OH), 4.52-4.13 (m, 5H), 2.63-2.52 (m, 2H), 2.39-2.04 (m, 3H), 1.81-1.75 (m, 5H), 1.67 (s, 3H), 1.62-1.55 (m, 2H), 1.39-1.35 (m, 4H), 0.91 (t, J=6.2 Hz, 3H); ¹³C {¹H}NMR (101 MHz, CDCl₃) δ 155.7, 154.9, 153.1, 147.5, 141.0, 139.3, 139.1, 135.1, 132.0, 124.7, 116.6, 115.5, 110.8, 109.4, 46.5, 37.4, 36.6, 36.5, 32.9, 32.0, 30.6, 30.3, 28.1, 23.7, 22.6, 19.4, 14.0; [α]_{D}²⁰=−39 (c=1.0, MeOH); HRMS (ESI-TOF) m/z: calcd for C₂₆H₃₆N₃O₂, 422.2808 [M+H]; found, 422.2789.

### Example 17: Biological Evaluation

Each embodiment of the current innovation is outlined in greater detail below:

### 17A. Cell Viability Assay in Neuronal Cell

In the present disclosure outlined herein, a focused library of cannabidiol analogues were studied for possible neuro-protective potential employing cell line-based stress model systems which mimic the biological stress conditions in vitro (J Cell Biochem., 2018, 119(8), 6482-6491).

Human neuroblastoma cells (SHSY5Y) and mouse neuroblastoma cells (N2A) were procured from American Type Culture Collection (ATCC). SHSY5Y cell cultures were maintained in DMEM F12 (Invitrogen) whereas N2A were cultured in DMEM (Invitrogen) with 10% Fetal Bovine Serum (Gibco) medium, subjected to differentiation to acquire neuronal morphology (employing differentiation media DMEM-F12, 3-5% FBS, (For differentiation, low concentration of FBS was used) and 50 μM retinoic acid). Differentiated cells were later seeded into 96 well plates, at a density of 1×10⁴ before being incubated for 48 hrs with the synthesized library. The exhausted culture media was substituted with new culture media containing 0.5 mg/ml MTT dye. Eventually, the OD at 490 nm excitation was measured using a 96-well plate reader. The proportion of viable cells was determined as percentage of absorbance of untreated cultures relative to control.

Among the screened library, most of the molecules maintained the cell viability at concentration of 20 μM, observed up to 48 hrs (results are summarized in Table 1A). Following molecules showed cytoprotective potential along with parent molecule B1 which was used as positive control: A1 (SHSY5Y-115.71±0.71: N2A-113.75±2.28); A3 (SHSY5Y-102.58±2.25: N2A-99.70±0.42); A7 (SHSY5Y-101.96±1.57: N2A-101.73±0.47); A11 (SHSY5Y-100.78±3.48: N2A-101.65±1.53); B1 (SHSY5Y-108.00±1.13: N2A-107.00±1.53).

TABLE 1A
Effect of CBD analogues on cell viability
in differentiated neural cells

| | | |
|---|---|---|
| S. | Test | % Cell viability ± SD |

| No | compounds | SHSY5Y | N2A |
|---|---|---|---|
| 1 | Control | 100 ± 0.00 | 100 ± 0.00 |
| 2 | A1 | 115.71 ± 0.71 | 113.75 ± 2.28 |
| 3 | A1′ | 93.64 ± 2.01 | 94.65 ± 1.86 |
| 4 | A2 | 95.83 ± 1.10 | 96.87 ± 3.77 |
| 5 | A3 | 102.58 ± 2.25 | 99.70 ± 0.42 |
| 6 | A4 | 97.28 ± 0.54 | 97.71 ± 0.81 |
| 7 | A5 | 94.46 ± 1.12 | 90.64 ± 1.85 |
| 8 | A6 | 27.03 ± 2.02 | 25.26 ± 1.47 |
| 9 | A7 | 101.96 ± 1.57 | 101.73 ± 0.47 |
| 10 | A8 | 89.41 ± 1.70 | 93.30 ± 0.39 |
| 11 | A9 | 91.67 ± 0.56 | 94.26 ± 1.30 |
| 12 | A10 | 98.08 ± 2.25 | 96.02 ± 2.17 |
| 13 | A11 | 100.78 ± 3.48 | 101.65 ± 1.53 |
| 14 | A12 | 95.92 ± 3.39 | 96.22 ± 2.32 |
| 15 | A13 | 96.15 ± 4.87 | 97.19 ± 3.51 |
| 16 | A14 | 90.50 ± 0.14 | 95.89 ± 4.04 |
| 17 | A15 | 81.79 ± 1.17 | 85.23 ± 1.25 |
| 18 | A16 | 86.970 ± 0.12 | 89.85 ± 0.03 |
| 19 | B1 | 108 ± 1.13 | 107 ± 1.56 |

### 17B. Cell Viability Assay in Macrophage in RAW264.7 Cells

RAW264.7 cells were seeded in 96-well cell culture plates at a density of 1×10⁴ cells per well and incubated overnight at 37° C. with 5% CO₂. Next day, the cells were treated with CBD or its derivatives at 5 μM and/or 10 μM concentration for 24 hrs. The MTT dye (2.5 mg/ml, 20 μL) was added to each well 4 hrs before termination (Molecules. 2022, 27, 8068). After 4 hrs of incubation with MTT, the supernatant was discarded, and the formazan crystals were dissolved by adding 100 μL DMSO. The absorbance was recorded at 540 nm on microplate reader (Tecan Infinite Pro).

All the compounds maintained the cell viability at the tested concentration. The results revealed that the indicated concentrations of most of the test compounds maintained the cell viability. The results are depicted in Table 1B.

TABLE 1B
Effect of CBD analogues on cell viability in RAW264.7 cells

| S. | Test | Concentration | % cell |
|---|---|---|---|
| No | compound(s) | (μM) | viability ± SD |
| 1 | Control | | 100 ± 0 |
| 2 | A1 | 5 | 93.1 ± 1.1 |
| | | 10 | 83.5 ± 0.6 |
| 3 | A1′ | 5 | 94.4 ± 0.9 |
| | | 10 | 92.4 ± 1.2 |
| 4 | A2 | 5 | 65.9 ± 6.5 |
| | | 10 | 61.7 ± 8.1 |
| 5 | A3 | 5 | 92.8 ± 0.9 |
| | | 10 | 89.9 ± 1.2 |
| 6 | A4 | 5 | 93.8 ± 1.14 |
| | | 10 | 90.6 ± 0.7 |
| 7 | A5 | 5 | 94.4 ± 1.3 |
| | | 10 | 90.4 ± 0.9 |
| 8 | A6 | 5 | 94.1 ± 1.8 |
| | | 10 | 92.0 ± 1.1 |
| 9 | A7 | 5 | 90.1 ± 1.3 |
| | | 10 | 93.0 ± 1.7 |
| 10 | A8 | 5 | 94.7 ± 2.7 |
| | | 10 | 87.1 ± 1.9 |
| 11 | A9 | 5 | 94.2 ± 2.1 |
| | | 10 | 87.6 ± 0.1 |
| 12 | A10 | 5 | 97.5 ± 1.7 |
| | | 10 | 95 ± 2.6 |
| 13 | A11 | 5 | 93.3 ± 0.4 |
| | | 10 | 91.6 ± 0.2 |
| 14 | A12 | 5 | 94.5 ± 1.0 |
| | | 10 | 92.7 ± 0.5 |
| 15 | A13 | 5 | 89.3 ± 2.0 |
| | | 10 | 93.9 ± 4.3 |
| 16 | A14 | 5 | 95.2 ± 0.7 |
| | | 10 | 89.8 ± 1.1 |
| 17 | A15 | 5 | 93.4 ± 1.1 |
| | | 10 | 89.9 ± 0.9 |
| 18 | A16 | 5 | 93.6 ± 1.3 |
| | | 10 | 87.7 ± 0.8 |
| 19 | B1 | 5 | 95.5 ± 1.5 |
| | | 10 | 91.0 ± 1.6 |

### 17C. In Vitro Neuro-Protection Activities

In the next attempt, the neuroprotective properties of the generated library (20 μM, 48 hrs) were assessed employing differentiated human neuroblastoma cell line SHSY5Y and mouse neuroblastoma cell line N2A (Biomol Ther., 2022, 30(6), 570-575). The cells were assaulted with the neurotoxic corticosterone (Cort, 400 μM) for 24 hrs after the overnight preincubation of cells with test molecules as a selection criterion. The results are summarized in Table 2A. The newly generated library showed better cell viability in comparison to corticosterone treated group. The cell viability was comparatively higher in case of following molecules and the highest number of viable cells were observed in A1 treated line. A1 (SHSY5Y-97.86±1.31: N2A-97.60±1.97); A3 (SHSY5Y-81.54±0.18): N2A-86.65±0.58); A7 (SHSY5Y-88.52±1.26: N2A-86.01±2.06); A11 (SHSY5Y-87.68±0.68: N2A-77.80±1.51); B1 (SHSY5Y-96.07±1.85: N2A-95.66±3.24).

TABLE 2A
Neuroprotective potential of novel CBD analogues after 48 hrs

| | | |
|---|---|---|
| S. | Test | % Cell viability ± SD |

| No | compound(s) | SHSY5Y | N2A |
|---|---|---|---|
| 1 | Control only | 100 ± 0.00 | 100 ± 0.00 |
| 2 | Cort. only | 56.31 ± 1.89% | 52.10 ± 2.78% |
| 3 | A1 + Cort. | 97.86 ± 1.31 | 97.60 ± 1.97 |
| 4 | A1′ + Cort. | 73.30 ± 2.73 | 79.17 ± 0.28 |
| 5 | A2 + Cort. | 87.19 ± 3.16 | 85.18 ± 3.83 |
| 6 | A3 + Cort. | 81.54 ± 0.18 | 86.65 ± 0.58 |
| 7 | A4 + Cort. | 71.80 ± 2.51 | 73.13 ± 1.94 |
| 8 | A5 + Cort. | 77.61 ± 1.90 | 75.30 ± 2.02 |
| 9 | A6 + Cort. | 17.45 ± 0.62 | 21.30 ± 0.66 |
| 10 | A7 + Cort. | 88.52 ± 1.26 | 86.01 ± 2.06 |
| 11 | A8 + Cort. | 84.30 ± 0.04 | 85.07 ± 0.20 |
| 12 | A9 + Cort. | 73.12 ± 2.15 | 78.84 ± 0.96 |
| 13 | A10 + Cort. | 66.12 ± 4.89 | 55.49 ± 2.88 |
| 14 | A11 + Cort. | 87.68 ± 0.68 | 77.80 ± 1.51 |
| 15 | A12 + Cort. | 76.85 ± 0.40 | 75.46 ± 0.02 |
| 16 | A13 + Cort. | 85.68 ± 1.05 | 85.80 ± 1.58 |
| 17 | A14 + Cort. | 83.35 ± 1.02 | 82.04 ± 1.08 |
| 18 | A15 + Cort. | 65.16 ± 2.51 | 67.67 ± 2.01 |
| 19 | A16 + Cort. | 76.77 ± 1.43 | 75.49 ± 4.29 |
| 20 | B1 + Cort. | 96.07 ± 1.85 | 95.66 ± 3.24 |

In another experiment, the cytoprotective effect of A1 as well as parent molecule B1 was determined in TrkB stable cells SHSY5Y. A1 exhibited increased cell viability versus control after a time span of 96 hrs and the results are depicted in Table 2B (Acta Pharm Sin B., 2021, 11(7), 1903-1913.

TABLE 2B
Neuro-protective potential of novel
CBD analogues in SHSY5Y after 96 hrs

| | | Concen- | | |
|---|---|---|---|---|
| S. | | tration | | % Cell |
| No | Group | (μM) | Type of group | viability ± SD |
| 1 | Control | — | vehicle | 100 ± 0.00% |
| 2 | Cort. | 400 | Negative control | 52.64 ± 0.71% |
| 3 | A1 | 20 | Invention molecule | 110.42 ± 1.61% |
| 4 | A1 + Cort. | 400 + 20 | Negative control + | 97.15 ± 1.13% |
| | | | invention molecule | |
| 4 | B1 | 20 | Parent molecule | 102.22 ± 1.06% |
| 5 | B1 + Cort. | 400 + 20 | Parent molecule + | 86.67 ± 1.16 |
| | | | Negative Control | |

In another experiment, differentiated SHSY5Y cell cultures subjected to A1 for extended duration of time (up to 96 hrs) with and without corticosterone revealed that A1 was non-toxic even at concentrations as high as 40 μM in both concentration and time dependent manner.

Treating cells with 1.25, 2.5, 5, 10, 20 and 40 μM of A1 molecule alone showed no significant variation in cell viability when compared to positive control DHF. Furthermore, A1 intervention in presence of corticosterone evidenced neuroprotective effect against corticosterone induced impairment and cell death. A1 exhibited a substantial level of neuroprotection by significantly lowering the corticosterone cytotoxic activity.

TABLE 2C
Neuro-protective potential of novel CBD analogues in SHSY5Y after 96 hrs

| | Cell viability | | | | | | | | |
|---|---|---|---|---|---|---|---|---|---|
| S. | Cort | DHF | A1 μM | | | | | | |
| No. | 400 μM | 5 nM | 40 | 20 | 10 | 5 | 2.5 | 1.25 | 0 |
| 1 | − | 113.7 ± | 115.4 ± | 115.3 ± | 117.15 ± | 108.46 ± | 108.46 ± | 102.15 ± | 100 ± |
| | | 0.44 | 2.50 | 3.62 | 3.24 | 1.92 | 1.69 | 0.29 | 0.00 |
| 2 | + | 104.3 ± | 102.1 ± | 107.6 ± | 112.66 ± | 102.95 ± | 93.64 ± | 83.81 ± | 52.64 ± |
| | | 1.22 | 1.76 | 1.37 | 0.66 | 0.66 | 2.77 | 4.63 | 0.71 |

Here, A1 was tested to find its role in eliciting survival pathway proteins in differentiated human neuroblastoma cells in concentration and time dependent manner.

To investigate the effect of A1 on the survival proteins, SHSY5Y cell cultures were treated with different concentrations for 6 hrs and analyzed for the expression of survival proteins, including p-Erk 44/42, p-AKT 473, Anti BDNF, and p TrkB 816 and p TrKB 515 by western blotting. The expression of p-Erk 44/42, Anti BDNF, and p TrkB 816, and p TrkB 515 was increased significantly except for the expression of p-AKT 473 and p TrkB 515. However, a decrease was observed in anti BDNF, p TrkB 816, p-Erk 44/42, p TrkB 515, and p-AKT 473 levels after corticosterone insult and the results are depicted in Table 2D (EMBO Mol Med. 2019 July; 11(7), e9950).

TABLE 2D
Effect of 1A on survival pathway proteins in SHSY5Y

| | Relative Protein Expression | | | | | |
|---|---|---|---|---|---|---|
| | Conc | p ERK | PAKT | Anti | p TrkB | p TrkB |
| | (μM) | 44/42 | 473 | BDNF | 816 | 515 |
| Control | — | 1.00 | 1.03 | 1.00 | 1.00 | 1.00 |
| Cort. | 400 | 0.36 | 0.01 | 0.10 | 0.36 | 0.15 |
| DHF | 5 nM | 5.28 | 1.42 | 1.03 | 1.55 | 2.43 |
| DHF/Cort. | — | 5.00 | 0.96 | 0.53 | 0.71 | 2.11 |
| A1 | 5 | 4.51 | 0.67 | 0.67 | 3.11 | 0.23 |
| A1/Cort. | | 3.70 | 0.60 | 0.44 | 1.41 | 0.07 |
| A1 | 10 | 8.80 | 1.60 | 1.48 | 2.91 | 1.75 |
| A1/Cort. | | 5.05 | 1.35 | 1.34 | 2.89 | 1.30 |

To evaluate the suppressive effects of corticosterone on cell survival pathway proteins, differentiated SHSY5Y cell cultures were exposed to A1 for multiple time spans which remarkably increased the expression of p-Erk 44/42, p-AKT 473, Anti BDNF, p TrkB 515 and p TrkB 816. These findings, therefore, demonstrate that A1 could suppress the corticosterone induced impairment in cell population in-vitro. The results are depicted in Table 2E.

TABLE 2E
Effect of 1A on relative expression of
survival pathway proteins in SHSY5Y

| | | Relative Protein expression | | | | |
|---|---|---|---|---|---|---|
| | Time | p ERK | PAKT | Anti | p TrkB | p TrkB |
| | (hr) | 44/42 | 473 | BDNF | 816 | 515 |
| A1 + | 0 | 1.00 | 1.00 | 1.029159 | 1.012302 | 1.018826 |
| Cort. | 0.5 | 1.32 | 11.68 | 14.02406 | 1.30879 | 2.276945 |
| | 1 | 3.29 | 14.78 | 28.11356 | 10.53982 | 6.569916 |
| | 2 | 8.33 | 17.15 | 51.75625 | 16.75889 | 8.706565 |
| | 4 | 9.69 | 26.33 | 41.62227 | 23.54123 | 8.144829 |
| | 6 | 10.62 | 31.80 | 39.43335 | 23.0422 | 6.675055 |

### 17D. In Vitro Anti-Inflammatory Activity:

Measurement of Nitric oxide: RAW264.7 macrophages were seeded in 96-well plates with a density of 0.5×10⁵ cells/well and incubated overnight at 37° C. in a 5% CO₂ atmosphere. The cells were treated with different concentration of CBD and its analogues and exposed to LPS for 24 hrs. For nitric oxide (NO) determination, the cell culture supernatant was taken and mixed with equal volume of Griess reagent in another 96 well plate at room temperature (Biomed Pharmacother., 2017, 92, 175-186). The absorbance was measured at 540 nm. The final calculations were determined on a NaNO₂ standard curve.

The maximum NO levels were observed in LPS exposed cells. The production of NO decreased in cells pre-treated with CBD and its analogues. A10 inhibited NO production more significantly than other derivatives, which was 68.3% and 48.9% at concentrations 10 μM and 5 μM, respectively. Other derivatives such as A11, A9 and A13 showed more than 25% inhibition, whereas A4 and A1′ showed greater than 20% inhibition. The results are depicted in Table 3A.

TABLE 3A
Effect on nitric oxide production

| S. | Test | Concentration | % NO |
|---|---|---|---|
| No | compound(s) | (μM) | Inhibition ± SD |
| 1 | Basal control | | 98.51 ± 1.0 |
| 2 | LPS | 1 μg/ml | 0 |
| 3 | A1 | 5 | 7.1 ± 0.8 |
| | | 10 | 12.3% |
| 4 | A1′ | 5 | 15.6 ± 2.5 |
| | | 10 | 23.4 ± 3.8 |
| 5 | A3 | 5 | 4.9 ± 1.4 |
| | | 10 | 10.7 ± 2.1 |
| 6 | A4 | 5 | 11.4 ± 2.9 |
| | | 10 | 20.8 ± 2.5 |
| 7 | A5 | 5 | 5.8 ± 2.4 |
| | | 10 | 12.3 ± 3.1 |
| 8 | A6 | 5 | 7.6 ± 3.0 |
| | | 10 | 11.8 ± 1.9 |
| 9 | A7 | 5 | 8.4 ± 3.1 |
| | | 10 | 16.0 ± 1.2 |
| 10 | A8 | 5 | 10.7 ± 2.3 |
| | | 10 | 19.6 ± 3.2 |
| 11 | A9 | 5 | 14.6 ± 3.3 |
| | | 10 | 27.9 ± 0.4 |
| 12 | A10 | 5 | 48.9 ± 1.3 |
| | | 10 | 68.3 ± 0.9 |
| 13 | A11 | 5 | 15.6 ± 1.7 |
| | | 10 | 31.4 ± 3.6 |
| 14 | A12 | 5 | 8.6 ± 0.93 |
| | | 10 | 11.4 ± 3.5 |
| 15 | A13 | 5 | 12.0 ± 1.7 |
| | | 10 | 27.3 ± 2.6 |
| 16 | A14 | 5 | 8.5 ± 1.7 |
| | | 10 | 14.2 ± 3.8 |
| 17 | A15 | 5 | 4.8 ± 0.02 |
| | | 10 | 14.5 ± 0.6 |
| 18 | A16 | 5 | 11.5 ± 1.2 |
| | | 10 | 18.3 ± 1.0 |
| 19 | B1 | 5 | 24.3 ± 3.2 |
| | | 10 | 38.3 ± 3.7 |

### 17E. In Vivo Efficacy (Ameliorates Behavioral Despair) in Mice Depression Model:

In vivo efficacy (ameliorates behavioral despair) of A1 in corticosterone induced model of stress (J. Neurochem., 2018 146, 722 734). To determine if A1 could attenuate behavioral distress in vivo, male C57 mice weighing 32 g (4-6-week-old) was employed, arbitrarily divided into 6 groups: control, Corticosterone, Fluoxetine, Fluoxetine+Corticosterone, A1, A1+Corticosterone and subjected them to chemically induced corticosterone stress for 21 days. A1 (20 mg/kg, oral) dissolved in normal saline was given for 3 consecutive weeks following corticosteroid stress. The sucrose preference test was employed, tail suspension test, and forced swim test to evaluate behavioral distress. We noticed that A1 can resurrect mice in stressful situations. Moreover, animals retained their health throughout the trail amidst insignificant weight fluctuations, implying that A1 had no discernible toxicity. Animals receiving corticosterone treatment exhibited a decreased appetite, resulting in a repercussion on their body weight. Mice exposed to stress for continuous period of 3 weeks showed significant drop in body weight (27.40±1.91) when compared to the control mice (42.08±2.49, p**<0.01). However, chronic A1 oral administration of 20 mg/kg for 21 days significantly increased the body weight in stressed group comparative to corticosterone only: (32.17±2.07, p**<0.01). The results are depicted in Table 4A.

TABLE 4A
In vivo efficacy in depression mice model (Effect on Body weight)

| Group | Dosage (mg/kg) | | Model group | Weight (g) ± SD |
|---|---|---|---|---|
| Control | 0.9% NaCl | | Control | 42.08 ± 2.49 |
| Cort. | 35 | μg/ml | Negative control | 29.57 ± 3.90 |
| Fluoxetine | 18 | mg/kg | Positive control | 40.42 ± 2.25 |
| Fluoxetine + | 35 μg/ml + | | Positive | 38 ± 2.16 |
| Cort. | 18 mg/kg | | control + | |
| | | | negative control | |
| A1 | 20 | mg/kg | Present invention | 42.88 ± 2.69 |

| | | | |
|---|---|---|---|
| A1 + Cort. | 20 mg/kg + | Present | 36.75 ± 3.88 |
| | 35 μg/ml | invention + | |
| | | Negative control | |

Afterwards, it was observed that A1 significantly increased sucrose consumption by more than 50%, comparable to stressed mice, influencing drug-induced depressed states. Sucrose consumption was employed to evaluate anhedonia, a core hallmark of depression in mice. The animals were allowed unfettered access to both identical bottles, one containing water while the other contained a 2% solution of sucrose, for 21 days.

The below equation was later employed to determine water and sugar consumed by mice:

Sucrose Preference=Sucrose consumed+water consumed/Total fluid intake.

Sucrose consumption was significantly lower in stressed mice (43.50±3.35) compared to control mice (82.8±2.46, p**<0.01, following A1 intervention alone sucrose upsurge was increased (83.80±0.80%), however A1 along with corticosterone subjected mice sucrose intake was more comparable to stressed mice (78.02±0.99, p**<0.01) The results are depicted in Table 4B.

TABLE 4B
In vivo efficacy in depression mice
model (Diet consumption behavior)

| | | % Sucrose consumption |
|---|---|---|
| Group | Model group | (ml) ± SD |
| Control | Control | 82.8 ± 2.46 |
| Cort. | Negative control | 43.50 ± 3.35 |
| Fluoxetine | Positive control | 82.10 ± 0.29 |
| Fluoxetine + | Positive | 72.39 ± 2.69 |
| Cort. | control + | |
| | negative control | |
| A1 | Present invention | 83.80 ± 0.80 |
| A1 + Cort. | Present | 78.02 ± 0.99 |
| | invention + | |
| | Negative control | |

Tail Suspension Test (TST) is a behavioral test employed to assess perturbations expected to influence depression like phenotypes. TST is grounded on the reflection that the extent of immobility is allied with depressing phenotype of mice overturned by antidepressants. Mice were positioned 2 cm from their tail tips and 5 cm above the ground suspending the tails using adhesive tape for the free movement (Acta Neuropathol Commun., 2016, 4(1), 108).

The contemporary immobility was alluded to as interruption in motion, where a blind assessor evaluates the disappointed aspect of display before scoring it. The mice under corticosterone-prompted stress appeared to have an extended dead time or immobility period (52.32±9.96 sec), with a significant decline in lag to motionless condition (6.97±4.52 sec) comparable to the immobility time in control (27.47±7.91 sec) with enhanced latency to motionless states in depressed mice (47.44±9.87 sec).

Next, the subsequent effect of A1 was evaluated in relation to corticosterone prompted cytotoxicity in mice. Mice were given 20 mg/kg of A1, which by itself demonstrated a significant drop-in dead time (28.76±9.01 sec) compared to the combinational treatment of A1 and corticosterone (33.05±5.62 sec, p**<0.01). A1 alone also depicted significant upsurge in lag to inactivity in mice (54.35±9.74 sec) which was comparable to the combinational treatment group (36.90±14.54 sec, p*<0.05). Treatment with agonist, Fluoxetine at cone of 18 mg/kg alone caused significant alternation in dead time (19.61±7.90) with prolonged latency to immobility (49.51±7.84 sec, p**<0.01) whereas the treatment with corticosterone (35 ug/ml) along with fluoxetine (18 mg/kg) significantly rescued mice from stressed conditions by boosting latency to immobility dead time (34.79±6.38 sec) with a decrease in dead time (35.83±12.71 sec) compared to corticosterone; p**<0.01). As a result, A1 could shorten the duration of stillness in corticosterone-treated mice, prolonging the time frame of latency before immobilization begins. The results are depicted in Table 4C.

TABLE 4C
In vivo efficacy in depression mice
model (Effect on % immobility)

| | | | % Latency to |
|---|---|---|---|
| | | % Immobility | immobility |
| Group | Model group | (sec) ± SD | (sec) ± SD |
| Control | Control | 27.47 ± 7.91 | 47.44 ± 9.87 |
| Cort. | Negative | 52.32 ± 9.96 | 6.97 ± 4.52 |
| | control | | |
| Fluoxetine | Positive control | 19.61± | 49.51 ± 7.82 |
| Fluoxetine + | Positive | 35.83 ± 12.71 | 34.79 ± 6.38 |
| Cort. | control + | | |
| | negative control | | |
| A1 | Present | 28.76 ± 9.10 | 54.35 ± 9.74 |
| | invention | | |
| A1 + Cort. | Present | 33.05 ± 5.62 | 36.90 ± 14.54 |
| | invention + | | |
| | Negative | | |
| | control | | |

The forced swim test was employed to evaluate behavioral melancholy in depressed subjects. Each animal was individually conditioned in a glass cylinder 20 cm long, 10 cm deep and 14 cm radius of curvature. After being submerged in water for 5 mins, the mice's cumulative stationary episode, struggle in water, and eventual floating condition were evaluated. The duration for which the animals were motionless while attempting to hold both of their front limbs still was calculated by interpreting the visualizations portraying behavioral despair (Front Behav Neurosci., 2018, 12, 42).

Corticosterone (35 μg/ml) treatment alone increased dead time (67.15±12.32 sec) when compared to placebo (26.94±10.26 sec, p*<0.05) with decline in latency to immobility in stressed mice (16.14±5.68 sec) comparable to control (41.94±14.82 sec, p**<0.01) A1, a corticosterone antagonist, significantly increased the lag phase (44.59±19.55 sec) in comparison to the corticosterone group (16.14±5.68 sec, p**<0.01) whereas A1 combined with corticosterone had a synergistic effect on immobility time (24.22±5.30 sec, p**<0.01) in comparison to the corticosterone group. Fluoxetine, positive control alone substantially enhanced the lag to immobility higher than the corticosterone group, suggesting related synergistic activity with corticosterone (55.34±15.71 sec; 16.14±5.68 sec, p**<0.01) Further, A1 and fluoxetine were able to significantly reverse the stasis when administered to mice in the presence of corticosterone (17.67±5.12 sec, p**<0.01; 47.84±10.33 sec, p*<0.05). The results are depicted in Table 4D.

TABLE 4D
In vivo efficacy in depression mice model (Forced Swim Test)

| | | | % Latency to |
|---|---|---|---|
| | | % Immobility | immobility |
| Group | Model group | (sec) ± SD | (sec) ± SD |
| Control | Control | 26.94 ± 10.26 | 41.94 ± 14.82 |
| Corticosterone | Negative control | 67.15 ± 12.32 | 16.14 ± 5.68 |
| Fluoxetine | Positive control | 22.39 ± 4.91 | 55.34 ± 15.71 |
| Fluoxetine + | Positive | 47.84 ± 10.33 | 34.06 ± 11.21 |
| Cort. | control + | | |
| | Negative control | | |
| A1 | Present invention | 24.22 ± 5.30 | 44.59 ± 19.55 |
| A1 + Cort. | Present | 17.67 ± 5.12 | 34.06 ± 14.03 |
| | invention + | | |
| | Negative control | | |

### ADVANTAGES OF THE PRESENT INVENTION

The present invention deals with the synthesis of novel 2′-{heterocyclyl(aryl/alkyl)methyl}-cannabidiol as anti-depression and anti-inflammatory agents. The present invention also discloses the new generation compounds with better pharmacological activity in comparison to cannabidiol (CBD). The present invention also discloses the new compound containing hetero-atom which will help to improve the drug-like properties and bio-availability.

What is claimed is:

1. A ring annulated cannabidiol compound of Formula A and stereoisomers thereof, 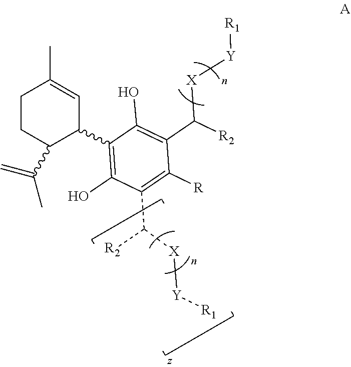
wherein X is independently selected from C or N;
Y is selected from heterocycle, or heteroaryl;
n and z are integers independently selected from 0 or 1;
R is selected from H, OH, protected hydroxyl, alkyl, alkenyl, alkynyl, acyl, aryl, heteroaryl, cycloalkyl or heterocycle, wherein the alkyl, alkenyl, alkynyl or acyl group is substituted with one or more groups, independently selected from the group consisting of halogen, —OH, alkyl, —O-alkyl, NR′R″, S-alkyl, alkynyl, aryl, heteroaryl, cycloalkyl and heterocycle; and aryl or heteroaryl group is substituted with one or more substituents selected from the group consisting of halogen, —OH, alkyl, —O-alkyl, —COOH, —C(O), —C alkyl, —C(O)OC and NR′R″; wherein R′ and R″ are independently selected from the group consisting of H, alkyl, alkenyl, alkynyl and acyl;
Ri is independently selected from the group consisting of H, OH, alkyl, alkenyl, alkynyl, acyl, aryl, heteroaryl, cycloalkyl and heterocycle, wherein the alkyl, alkenyl, alkynyl and acyl group is substituted with one or more groups, independently selected from the group consisting of halogen, —OH, alkyl, —O-alkyl, NR′R″, S-alkyl, —SO-alkyl, —SO2-alkyl, aryl, S-aryl, —SO-aryl, —SO2-aryl, —SO2-N-aryl, -A-SO2-arylalkenyl and alkynyl; aryl, heteroaryl, cycloalkyl and heterocycle is substituted with one or more substituents selected from the group consisting of halogen, OH, alkyl, —O-alkyl, —COOH, —C(O), —C alkyl, —C(O)OC, NR′R″; wherein R′ and R″ are independently selected from the group consisting of H, alkyl, alkenyl, alkynyl and acyl; and
R2 is selected from H, alkyl, alkyl aryl and aryl.

2. The compound as claimed in claim 1, wherein Y is selected from the group consisting of morpholinyl, piperidinyl, piperazinyl, pyrazinyl, pyrrolidinyl, tetrahydrofuranyl, pyridinyl, pyrimidinyl, phenyl and benzyl.

3. The compound as claimed in claim 1, wherein Ri is selected from the group consisting of:

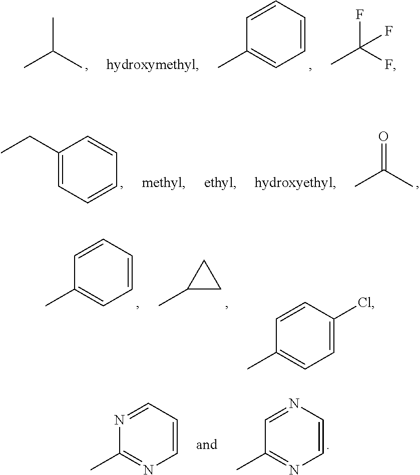

4. The compound as claimed in claim 1, wherein the compound is selected from the group consisting of;

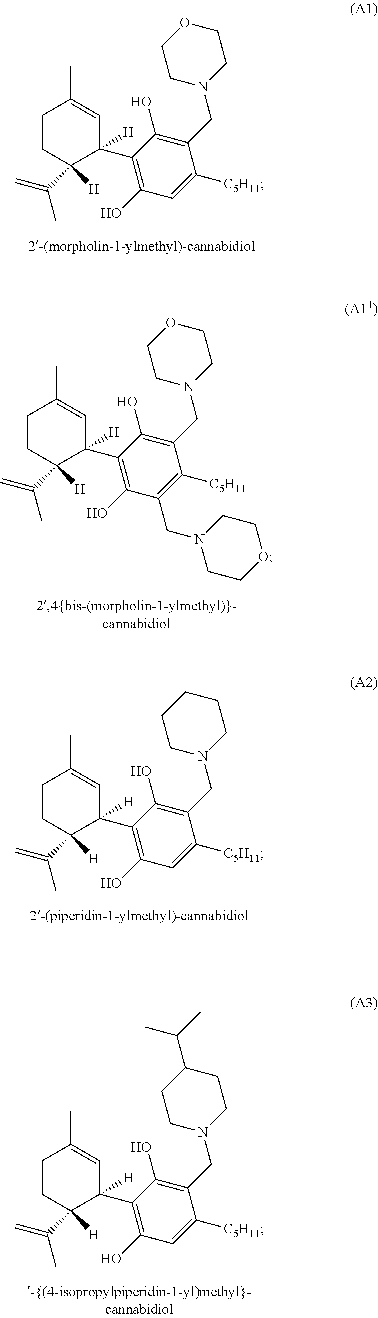

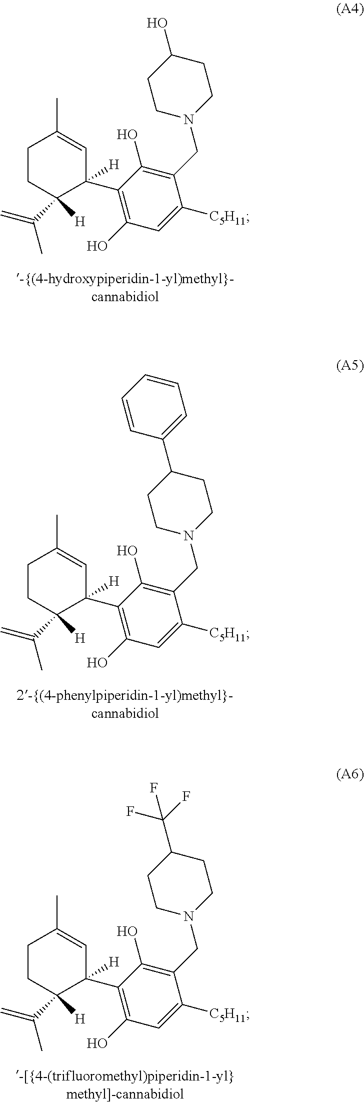

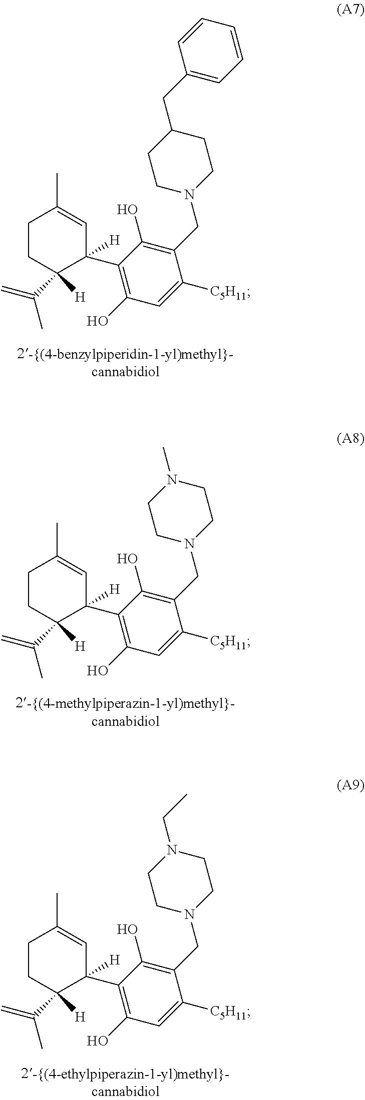

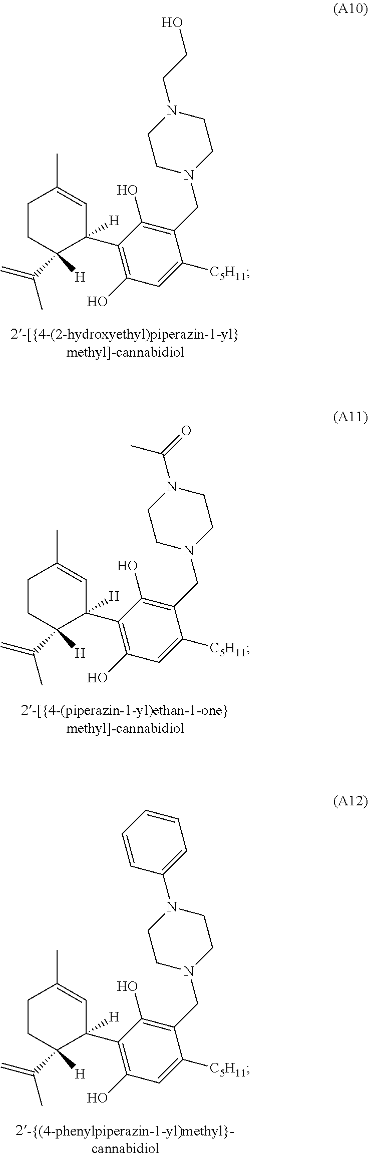

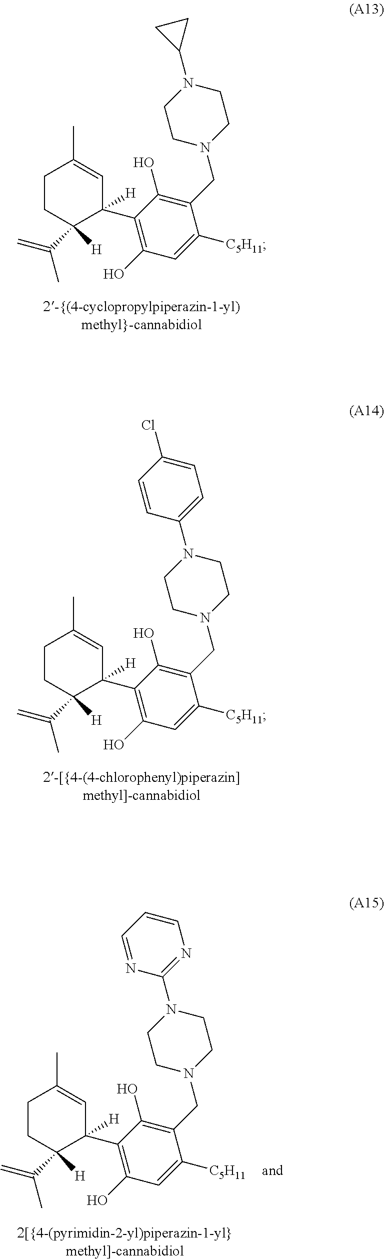

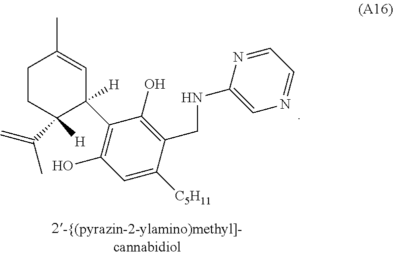

5. The compound as claimed in claim 1, wherein the compound has two stereo-centres which may be R, S or a mixture of both.

6. A process for the synthesis of ring annulated cannabidiol compound of Formula A and stereoisomers thereof, 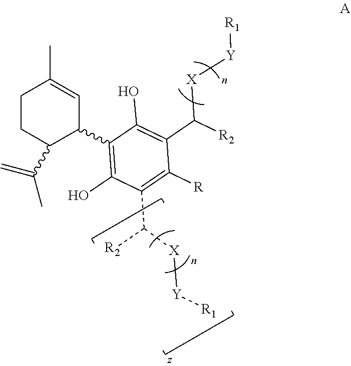
wherein X is independently selected from C or N;
Y is selected from heterocycle or heteroaryl;
n and z are integers independently selected from 0 or 1;
R is selected from H, OH, protected hydroxyl, alkyl, alkenyl, alkynyl, acyl, aryl, heteroaryl, cycloalkyl or heterocycle, wherein the alkyl, alkenyl, alkynyl or acyl group is substituted with one or more groups, independently selected from the group consisting of halogen, —OH, alkyl, —O-alkyl, NR′R″, S-alkyl, alkynyl, aryl, heteroaryl, cycloalkyl and heterocycle; and aryl or heteroaryl group is substituted with one or more substituents selected from the group consisting of halogen, —OH, alkyl, —O-alkyl, —COOH, —C(O), —C alkyl, —C(O)OC and NR′R″; wherein R′ and R″ are independently selected from the group consisting of H, alkyl, alkenyl, alkynyl and acyl;
Ri is independently selected from the group consisting of H, OH, alkyl, alkenyl, alkynyl, acyl, aryl, heteroaryl, cycloalkyl and heterocycle, wherein the alkyl, alkenyl, alkynyl and acyl group is substituted with one or more groups, independently selected from the group consisting of halogen, —OH, alkyl, —O-alkyl, NR′R″, S-alkyl, —SO-alkyl, —SO₂-alkyl, aryl, S-aryl, —SO-aryl, —SO₂-aryl, —SO₂—N-aryl, -A′-SO₂-arylalkenyl and alkynyl; aryl, heteroaryl, cycloalkyl and heterocycle is substituted with one or more substituents selected from the group consisting of halogen, OH, alkyl, —O-alkyl, —COOH, —C(O), —C alkyl, —C(O)OC, NR′R″; wherein R′ and R″ are independently selected from the group consisting of H, alkyl, alkenyl, alkynyl and acyl;
R2 is selected from H, alkyl, alkyl aryl and aryl. comprising the steps of:
i. coupling of fragment B, wherein R is as defined above and fragment C, wherein R₁ is as defined above in the presence of an aldehyde D, wherein R2 is as defined above at a temperature in the range of 25-30° C., for a period in the range of 13-16 hours and 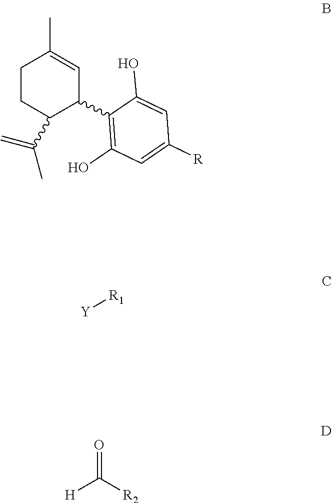
ii. purifying the obtained compound of formula A.

7. The process as claimed in claim 6, wherein the solvent is selected from the group consisting of dichloromethane, chloroform, isopropanol, acetone, acetonitrile methanol or a combination thereof.

8. The process as claimed in claim 6, wherein compounds of fragment C is selected from the group consisting of:

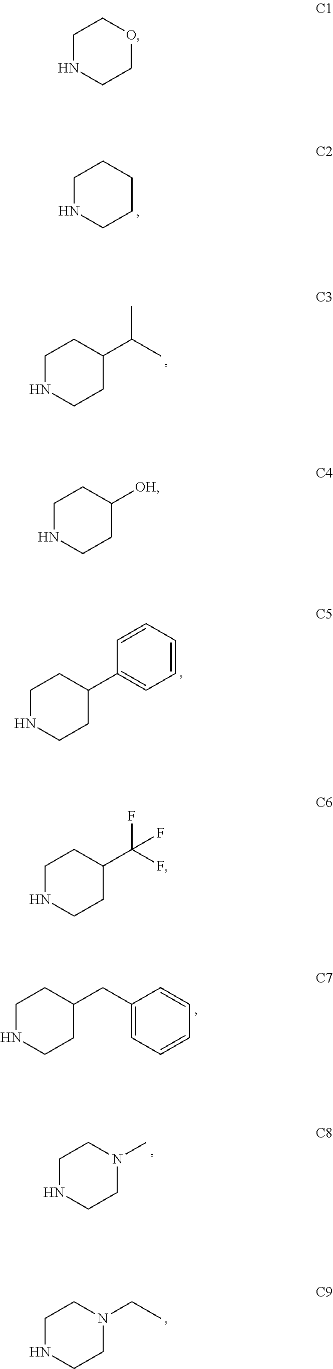

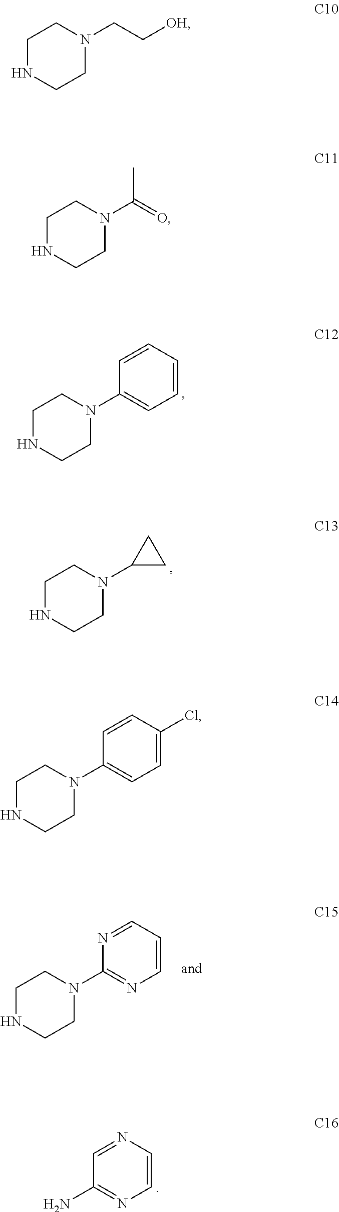

9. The process as claimed in claim 6, wherein the aldehyde D is selected from the group consisting of formaldehyde, propionaldehyde, acetaldehydes, propionaldehyde, butyraldehyde, isovaleraldehyde, benzaldehyde and cinnamaldehyde.

10. The compound as claimed in claim 1, wherein the compound exhibits neuroprotective properties, enhances the expression of survival pathway proteins, exhibits anti-inflammatory activity by inhibiting the NO production and exhibits cell viability potential in macrophages.
